(19) United States
(12) Patent Application Publication
(10) Pub. No.: US 2020/0022964 A1
(43) Pub. Date: Jan. 23, 2020

(54) COMPOSITIONS AND METHODS FOR TREATMENT OF INFLAMMATORY AUTOIMMUNE DISEASES

(71) Applicant: New York University, New York, NY (US)

(72) Inventor: Chuanju Liu, Orange, CT (US)

(21) Appl. No.: 16/518,494

(22) Filed: Jul. 22, 2019

Related U.S. Application Data
(60) Provisional application No. 62/701,806, filed on Jul. 22, 2018.

(51) Int. Cl. A61K 31/445; A61K 38/17; A61K 39/395; A61P 29/00

(52) U.S. Cl. CPC A61K 31/445; A61P 29/00; A61K 39/3955; A61K 38/1793

(57) ABSTRACT

Provided are compositions and methods for treatment of inflammatory autoimmune diseases. The composition comprises fexofenadine or terfenadine, and may further comprise another TNF-α activity inhibitor. The method comprises administering to a subject in need of treatment a therapeutically effective amount of fexofenadine, and optionally another TNF-α activity inhibitor.

### CROSS-REFERENCE TO RELATED APPLICATIONS

This application claims priority to U.S. Provisional Application No. 62/701,806, filed on Jul. 22, 2018, the disclosure of which is incorporated herein by reference.

### STATEMENT REGARDING FEDERALLY SPONSORED RESEARCH OR DEVELOPMENT

This invention was made with government support under grant numbers AR061484, AR062207, and NS070328 awarded by the National Institutes of Health. The government has certain rights in the invention.

### BACKGROUND OF THE DISCLOSURE

Autoimmune diseases are a series of disorders and conditions caused by immune intolerance to self-antigens which attack specific target organs and display diverse clinical signs. Inflammatory arthritis is the most common autoimmune disease, affecting about 1% of the population (Surolia et al., Nature 2010; 466:243-7). Autoimmune diseases are chronic diseases with complicated pathology and diverse clinical signs, underlying which are alterations in cytokine expression and immune cell infiltration. Among the proinflammatory cytokines involved, tumor necrosis factor alpha (TNF-α) has received considerable attention due to involvement in the pathogenesis of various disease processes, such as autoimmune disorders. TNF-α inhibitors (TNFI), including etanercept (Enbrel), infliximab (Remicade) and adalimumab (Humira), have been accepted as effective anti-inflammatory therapies and are among the most successful biotech pharmaceuticals. Although treatment with TNFI is effective in ameliorating disease in some patients, current TNFI fail to provide effective treatment for up to 50% of patients (Nurmohamed et al., Drugs 2005;65:661-94; Smolen et al., Ann Rheum Dis 2008; 67:1497-8). In addition to high cost (upwards of US $20,000 per year per patient using anti-TNF biologics), available TNFI have been found to contribute to infection risk in some patients (Kim et al., Nat Rev Rheumatol 2010; 6:165-74) and are associated with a slight increased risk of squamous cell cancer in rheumatoid arthritis patients treated with TNFI (Chen et al., Cytokine 2018; 101:78-88). Thus, there is an ongoing need to identify novel, safer and more cost-effective antagonists of TNF-α, such as antagonists with different inhibitory properties.

### SUMMARY OF THE DISCLOSURE

The present disclosure provides compositions and methods for treatment of inflammatory autoimmune diseases. Data is provided to demonstrate that Fexofenadine and Terfenadine inhibit TNF-α activity, and inhibit NF-κB nuclear translocation and activity. Our data indicate Fexofenadine and Terfenadine inhibit TNF-α via activation of cPLA2. Data is also provided to demonstrate therapeutic effects of Fex and Terf on inflammatory diseases in animal models.

In an aspect, this disclosure provides a method for treating an inflammatory autoimmune disease, such as, for example, inflammatory bowel disease or rheumatoid arthritis, comprising administering to a subject in need of treatment, a composition comprising a therapeutically effective amount of Fexofenadine and/or Terfenadine. In an embodiment, the disclosure provides a combination therapy comprising administering fexofenadine (or terfenadine) and another inhibitor of TNF-α. For example, the combination therapy may comprise administration of fexofenadine and Etanercept, Infliximab, Adalimumab, Certolizumab, or Golimumab.

In an aspect, the disclosure provides compositions comprising fexofenadine (or terfenadine) and one of Etanercept, Infliximab, Adalimumab, Certolizumab, or Golimumab, wherein the fexofenadine (or terfenadine) and the Etanercept, Infliximab, Adalimumab, Certolizumab, or Golimumab may be provided at therapeutic or sub-therapeutic doses.

In an aspect, the disclosure provides kits for treatment of inflammatory autoimmune diseases comprising in the same or different packaging, fexofenadine (or terfenadine) and the Etanercept, Infliximab, Adalimumab, Certolizumab, or Golimumab. The components may be provided as dry powders (such as lyophilized materials) or as liquid formulations, together with suspension liquids—if provided in a dry form, and optionally instructions materials.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1. Fexofenadine acts as an antagonist of TNF-α and inhibits TNF-α signaling and activity. (A) The molecular structure of fexofenadine (FFD) and terfenadine (TFD). CYP3A4, the major enzyme responsible for the metabolic process, is indicated. (B) BMDMs were treated without or with (10 ng/mL) in absence or presence of FFD (10 μM) for 24 hours. Total RNA was extracted for RNA-seq. A few typical TNF-α inducible genes that were suppressed by FFD were presented. (C) Transcription factor enrichment analysis from RNA-seq results, indicating the decreased gene expressions resulted from the suppressed activity of transcription factors NF-κB1 and RELA by FFD. (D-F) BMDMs were treated with or without TNF-α (10 ng/mL) in absence or presence of FFD (1 μM, 10 μM)/TFD (0.1 μM, 1 μM) for 24 hours. mRNA expressions of IL-1β, IL-6 and Nos-2 were tested by qRT-PCR. (G-H) BMDMs were treated without or with TNF-α (10 ng/mL) in absence or presence of FFD (1 μM, 10 μM)/TFD (0.1 μM, 1μM) for 48 hours. The levels of IL-1β and IL-6 in supernatant were detected by ELISA. (I) BMDMs were treated with M-CSF (10 ng/mL) for 3 days, then cultured with receptor activator of nuclear factor kappa-B ligand (RANKL) (100 ng/mL) and TNF-α (10 ng/mL) with or without FFD (10 μM) or TFD (1 μM) for 4 days and tartrate-resistant acid phosphatase (TRAP) staining was performed. Scale bar, 100 μm. (J) TNF-tg/NF-κB-Luc mice were applied to examine the anti-TNF effects of FFD/TFD in vivo. After FFD (2 or 10 mg/kg) and TFD (10 or 50 mg/kg) were orally administrated for 7 days, luciferase signals were detected by IVIS system. (K) BMDMs were treated with TNF-α (10 ng/mL) in the absence or presence of FFD (10 μM)/or TFD (1 μM) for various time points, as indicated. Cytoplasmic extraction (CE) and nuclear extraction (NE) were examined by Western blot with anti-p65 antibody. (L) BMDMs were cultured with TNF-α (10 ng/mL) in the absence or presence of FFD (10 μM) or TFD (1 μM) for 6 hours. Immunofluorescence cell staining was performed to visualize the subcellular localization of p65. 4′,6-diamidino-2-phenylindole (DAPI) was used to stain the nucleus. Scale bar, 25 μm, (M) p65 DNA binding activity was tested by ELISA. Excess amounts (100×) of wildtype (WT) and mutant oligo were used as positive and negative control, respectively (*p<0.05, **p<0.01, ***p<0.001). BMDMs, bone marrow-derived macrophages; qRT-PCR, quantitative real-time PCR; TNF-α, tumor necrosis factor alpha; TNF-tg, TNF-α transgenic.

FIG. 2. Fexofenadine prevents the spontaneous development of inflammatory arthritis in TNF transgenic mice. (A-H) TNF-tg mice (n=6) were orally administered fexofenadine (FFD, 10 mg/kg), terfenadine (TFD, 50 mg/kg) or methotrexate (MTX, 2 mg/kg, serving as a positive control) daily beginning at 8 weeks of age and continuing for a total of 13 weeks. During this period, treatment was halted at 17-week point indicated by red arrow and resumed at 19-week point indicated by green arrow. (A) Representative images of front paws and hind paws. (B-C) Swelling score. (D) H&E staining and quantification of histological score of knee and ankle samples. (E) TRAP staining of paw and skull samples. (F) Safranin O staining of knee and ankle samples. (G-H) Serum levels of IL-1β and IL-6, assayed by ELISA. (I-J) Therapeutic effects of FFD/TFD were tested by treating the TNF-tg mice with average swelling score reached around eight points (n=6). Swelling scores were recorded weekly (*p<0.05, **p<0.01, ***p<0.001) (scale bar, 100 μm). TNF-tg, TNF-α transgenic.

FIG. 3. Fexofenadine prevents the onset and progression of collagen-induced arthritis. (A-J) Collagen-induced arthritis (CIA) model of DBA/1J mice was used to test prevention effects of fexofenadine (FFD) and terfenadine (TFD) (n=8). FFD (10 mg/kg), TFD (50 mg/kg) and MTX (2 mg/kg) were orally delivered daily beginning 18 days after immunization. (A) The representative images of front paws and hind paws. (B) Paw thickness. (C) Clinical score of CIA. (D) The incidence rate of arthritis. (E) H&E staining and quantification of histological score of ankle samples. (F) TRAP staining of ankle samples. (G) micro CT of ankles. (H) Safranin O staining of ankle samples. (I-J) The serum levels of IL-1β and IL-6 in CIA models. (K-P) To examine the dosage-dependent therapeutic effects of FFD/TFD, CIA mice were treated with various dose of FFD or TFD, as indicated. FFD, TFD, MTX and vehicle were delivered after the clinical score reached approximately five points. (K) The clinical score of FFD-treated mice. (I) The clinical score of TFD-treated mice. (M-P) The serum levels of IL-1β and IL-6 (n=8) (*p<0.05, **p<0.01, ***p<0.001) (scale bar, 100 μm). MTX, methotrexate.

FIG. 4. Fexofenadine's anti-TNF activity is Histamine 1 receptor 1 (H1R1) independent. (A-B) The anti-TNF activity of terfenadine (TFD) and fexofenadine (FFD) does not depend on H1R1. (A) Immunoblotting analysis to examine the knockdown efficacy of siRNA against H1R1. (B) RAW264.7 cells transfected with scrambled control siRNA (scRNAi) or H1R1 RNAis were treated with or without TNF-α (10 ng/mL) in absence or presence of FFD (10 μM)/TFD (1 μM) for 48 hours. The levels of IL-1β and IL-6 in the medium were detected by ELISA. (C-D) Comparison of the anti-TNF activity between terfenadine (TFD)/fexofenadine (FFD) and other known H1R1 inhibitors. BMDM cells were treated without or with TNF-α (10 ng/mL) in absence or presence of various H1R1 inhibitor, as indicated, for 48 hours. The levels of IL-1β and IL-6 in medium were detected by ELISA. (E-G) Terfenadine (TFD) and fexofenadine (FFD) do not affect the binding of TNF-α and TNFR1 and to the cell surface. (E) Solid phase binding was used to reveal the dose-dependent binding of TNF-α to TNFR1. (F) The binding of TNF-α to TNFR1 in the presence of DMSO (negative control), FFD or TFD was also analyzed by solid phase binding. (G) RAW264.7 cells were incubated with biotin-labelled TNF-α in the absence or presence of TNF antibody (positive control), FFD (10 μM) or TFD (1 μM) for overnight, then cells were analyzed by flow cytometry (*p<0.05, **p<0.01, ***p<0.001). TNF-α, tumor necrosis factor alpha.

FIG. 5. cPLA2 is a novel target of fexofenadine. (A) Silver staining of DARTS assay. (B) Coomassie blue staining of DARTS assay. The band with molecular weight around 80 kDa protected by fexofenadine (FFD)/terfenadine (TFD) was indicated by arrow. (C) Adapted image of a mass spectra for PLA2G4A, encoding cPLA2. VLGVSGSQSR is SEQ ID NO: 11. (D) DARTS and Western blot to confirm FFD/TFD's binding targets. (E) CETSA melt response and associated curve. (F) Isothermal dose response (ITDR) and its curve. (G) IFD simulated binding complexes of cPLA2-FFD and cPLA2-TFD, respectively. cPLA2 is shown by surface representation (grey). FFD and TFD are shown by CPK representation with the atoms colored as carbon-violet (FFD) or cyan (TFD), oxygen-red, nitrogen-blue, hydrogen-white (only polar hydrogens of ligand are shown). (H-I) Docked poses of FFD and TFD in cPLA2, respectively, predicted by IFD. FFD and TFD are shown as ball and stick model with the same atom color scheme. Important amino acids are depicted as sticks with the same color scheme except that carbon atoms are represented in grey. Only polar hydrogens are shown. Dotted yellow lines indicate hydrogen-bonding interactions. Values of the relevant distances are given in A. (J) DARTS assay with serial deletion constructs encoding Flag-tagged mutants of cPLA2. cPLA2 (aa 1-750), cPLA2 (aa 126-750), cPLA2 (aa 406-750), cPLA2 (aa 1-479), cPLA2 (aa 1-144). 293 T cells were transfected with Flag-tagged mutants of cPLA2 plasmids, as indicated. DARTS assay samples were detected by Flag antibody. (K) DARTS assay for Ser-505 point mutant of cPLA2. The Ser-505 of cPLA2 was substituted with Ala 505. 293 T cells were transfected with the point mutant plasmid and DARTS was performed. Point mutated cPLA2 was detected by Flag antibody. CETSA, cellular thermal shift assay; cPLA2, cytosolic phospholipase A2; DARTS, drug affinity responsive target stability; IFD, induced-fit docking. In (K), 5′-TCT TAT CCA CTG TCT CCT TTG AGT GAC TTT-3′ is SEQ ID NO:9, and 5′-TCT TAT CCA CTG GCT CCT TTG AGA GAC TTT-3′ is SEQ ID NO:10.

FIG. 6. Fexofenadine inhibits TNF activity through binding to the catalytic domain 2 of cPLA2 and inhibition of the phosphorylation of cPLA2 on Ser-505. (A) Fexofenadine (FFD) inhibits the phosphorylation of cPLA2 on Ser-505. BMDM cells were treated with TNF-α (10 ng/mL) in the absence or presence of FFD (10 μM) for various time points, as indicated. p-p38, t-p38, p-ERK1/2, t-ERK1/2, p-cPLA2 (specifically for phosphorylated Ser-505), t-cPLA2 were detected by Western blot with corresponding antibodies. (B) Fexofenadine inhibits TNF-induced cPLA2 activity in living cells. RAW264.7 cells transfected with an expression plasmid encoding cPLA2 were treated with TNF-α and ATK, or terfenadine (TFD), or FFD overnight. Cells lysate was used for cPLA2 activity analysis. (C) FFD inhibits TNF-induced arachidonic acid (AA) production. The AA levels in BMDMs without or with TNF-α (10 ng/mL) in absence or presence of FFD or TFD for 48 hours were examined using a commercial ELISA kit. ATK was used as a positive control. (D-E) Addition of AA abolished FFD inhibition of TNF-induced cytokine release. BMDMs were treated with TNF-α (10 ng/mL), AA (10 μM) and FFD (10 μM)/TFD (1 as indicated. The levels of IL-1(3 and IL-6 were detected by ELISA. (F) Knockout efficiency of cPLA2 using CRISPR-Cas9 technique in RAW264.7 cells, assayed by Western blot. Two individual knockout clones (KO1 and KO2) were employed. (GH) Deletion of cPLA2 abolished FFD inhibition of TNF-induced cytokine release. WT and cPLA2 KO RAW264.7 cells were treated without or with TNF-α (10 ng/mL) in absence or presence of FFD (10 μM)/TFD (1 μM) for 48 hours. The levels of IL-1(3 and IL-6 in medium were detected by ELISA (*p<0.05, **p<0.01, ***p<0.001). (I) A proposed model for explaining the anti-TNF activity of FFD through targeting cPLA2 pathway. ATK, arachidonyl trifluoromethyl ketone 27; cPLA2, cytosolic phospholipase A2; TNF-α, tumor necrosis factor alpha.

FIG. 7. The 1st round screen of FDA approved drugs. THP-1 cell line with NF-κB beta-lactamase reporter gene stably integrated was used. The first screening was repeated 3 times. a. Schematic of the first screening using THP-1 cell line. Cells were incubated overnight with drugs (10 μM) followed by incubation with TNF-α (10 ng/ml) for 6 hours. Beta-lactamase reporter gene activity was measured. b. The analysis of the first round screen. c. Identities of twenty-four candidate drugs isolated from 1st round screen.

FIG. 8. The 2nd round screen of FDA approved drugs. a. The diagram of the second screening. NF-κB luciferase reporter plasmid was transfected into RAW 264.7 cells to confirm the results of the first-round screening. Cells were treated with drugs (10 μM) overnight, followed by treatment with TNF-α (10 ng/ml) for 6 hours, bioluminescence was taken as a measure of luciferase activity. b. The analysis of the second screening. TFD and FFD indicate Terfenadine and Fexofenadine, respectively. c. Identities of eight candidate drugs isolated from 2nd round screen.

FIG. 9. The 3rd round screen of FDA approved drugs. TNF-tg:NF-κB-Luc mice were generated to confirm the in vivo activity of 8 drugs isolated in preliminary screens. After treatment for 7 days with indicated compounds, the luciferase reporter signal was detected by IVIS. The drugs that show anti-TNF activity in vivo and the change of luciferase intensity at mouth and tail are highlighted in Red and Red Squares, respectively (Top panel). Terfenadine and Fexofenadine are underlined. The quantitative analysis for luciferase intensity (Bottom panel).

FIG. 10. RNAseq analysis. BMDMs were treated without (Ctrl) or with TNF-α (10 ng/ml) in absence or presence of Fexofenadine (FFD) (10 μM) or Terfenadine (TFD) (1 μM) for 24 hours. Total RNA were extracted for RNAseq. The genes which were induced by TNF-α and inhibited by both Terfenadine and Fexofenadine were summarized and analyzed.

FIG. 11. Terfenadine and Fexofenadine inhibit TNF-α activity in RAW264.7 cells. RAW264.7 cells were incubated with TNF-α (10 ng/ml) in the absence or presence of various dosages of Terfenadine (TFD) or Fexofenadine (FFD), as indicated, for 24 hours prior to collection for real time PCR or for 48 hours prior to collection for ELISA. a. The mRNA level of IL-1β, IL-6 and Nos-2 were detected by qRT-PCR. b. The secretion level of IL-1β and IL-6 was tested by ELISA. (* p<0.05, ** p<0.01, ***p<0.001)

FIG. 12. Terfenadine and Fexofenadine exhibit anti-TNF activity in BMDMs isolated from TNF transgenic mice. BMDMs were first incubated with M-CSF (10 ng/ml) for 6 days, Fexofenadine (FFD, 1 μM, 10 μM) or Terfenadine (TFD, 0.1 μM, 1 μM) were added for 24 hours for real time PCR or 48 hours for ELISA. a. The mRNA expression of IL-1β, IL-6 and Nos-2 were detected by qRT-PCR. b. The levels of IL-1β and IL-6 in medium were tested by ELISA. (*p<0.05, **p<0.01, ***p<0.001)

FIG. 13. BMDMs were treated with M-CSF (10 ng/ml) for 3 days, then cultured with RANKL (100 ng/ml) with or without FFD (10 μM) or TFD (1 μM) for 4 days and TRAP staining was performed. Scale bar, 100 μm.

FIG. 14. a. RMSD trajectory of cPLA2 and Fexofenadine in the cPLA2-Fexofenadine complex over the 10 ns MD simulation. b. RMSD trajectory of cPLA2 and Terfenadine in the cPLA2-terfenadine complex over the 10 ns MD simulation.

FIG. 15. ATK inhibits TNF-α induced cytokine release to a lesser degree when compared to Fexofenadine (FFD) and Terfenadine (TFD), shown in a. BMDM cultured in 6-well plates were treated with TNF-α with or without FFD, TFD and ATK for 48 hours. Similar dosage as used in FIG. 6c. The levels of IL-1β and IL-6 in medium were measured by ELISA. FFD and TFD inhibit TNF-α-, but not AA-, activated NF-κB reporter gene, shown in b. THP-1 cells bearing NF-κB reporter gene were cultured in 96-well plates. Cells were treated with FFD or TFD overnight and then TNF-α or AA were added and incubated for 6 hours. Similar dosage as used in FIG. 6d, e. The reporter gene activity was then measured. c. Control and cPLA2 knockout Raw264.7 cells were transfected with vector, cPLA2 or cPLA2 S505A (cPLA2 plasmid along with NF-κB luciferase reporter plasmid and renilla plasmid for 24 hrs, then the cells were treated with different doses of drug overnight, followed by treatment with TNF-α (long/ml) for 6 hours, bioluminescence was taken as a measure of luciferase activity.

FIG. 16. The effect of Fexofenadine (FFD) and Terfenadine (TFD) on macrophage in vitro (a, b). BMDMs were treated with M-CSF, IFN-γ (25 ng/ml) and LPS (250 ng/ml) or IL-4 (20 ng/ml) for polarization to type 1 macrophages (M1) or type 2 macrophages (M2) respectively. Various amounts of FFD or TFD, as indicated, were added. a. qPCR analysis of Il6, Nos2 mRNA expression in BMDMs polarized to M1 macrophages. b. qPCR analysis of Arg1 or Mgl1 mRNA expression in BMDMs polarized to M2 macrophages. c. The effect of Fexofenadine (FFD) and Terfenadine (TFD) on macrophages in vivo. Frozen splenic tissue specimens from TNF-α Tg mice treated with or without TFD or FFD (see FIG. 2 for detail) were stained and analyzed with dual immunofluorescence. All sections were permeabilized with 0.1% NP40 in PBS, and then blocked in 5% donkey serum for 30 min at RT, followed by double staining with anti-CD68 and iNOS (M1) or CD206 (M2). DAPI was used to stain the nuclei. The fluorescence intensity of cells was quantified by Image J software. (**p<0.01, ***p<0.001)

FIG. 17. The effects of Fexofenadine (FFD) and Terfenadine (TFD) on T cell differentiation, shown in a. The CD4+ T cells were isolated from spleen cells of wild-type 8-12 week old C57BL/6 mice to determine the differential induction of T cell subtypes in the presence of various amounts of FFD or TFD, as indicated, for 4 days. Flow cytometry was performed to examine the differentiation of Th1, Th2, Th17 and Treg cells. b. The effects of Fexofenadine (FFD) and Terfenadine (TFD) on splenic T cell composition in vivo. Frozen splenic tissue specimens from TNF-α Tg mice treated with or without TFD or FFD were stained and analyzed by dual immunofluorescence. All sections were permeabilized with 0.1% NP40 in PBS, and then blocked in 5% donkey serum for 30 min at RT, followed by incubation with anti-IFNg (Th1), anti-IL-4 (Th2), anti-IL17 (Th17) and anti-foxp3 (Treg) diluted in 5% donkey serum at 4° C. overnight. After being washed 3 times in PBS, the sections were incubated with Cy2-conjugated donkey anti-rat IgG for 30min at RT followed by washing., The sections were furthered stained with FITC conjugated anti-CD4 for 1 hour at RT. DAPI was used to stain the nuclei. The fluorescence intensity of cells was quantified by Image J software. (*p<0.05, **p<0.01, ***p<0.001).

FIG. 18. Effect of etanercept/fexofenadine combination in collagen-induced arthritis (CIA) mice. Mice were immunized with CII in CFA and boosted 21 days later to induce CIA, and the prevention started at 0 day after the second immunization. CIA mice with arthritis severity score above 5 after the second immunization were selected and treated (i.p. injection) with PBS, etanercept (5 mg/kg), etanercept (1 mg/kg) or fexofenadine(5 mg/kg)+etanercept (1 mg/kg). Results are shown for the prevention model (A) and the therapeutic model (B).

### DESCRIPTION OF THE DISCLOSURE

The present disclosure provides compositions and methods for targeting cytosolic phospholipase A2. The present compositions and methods can be used for treatment of conditions in which cPLA2 is implicated. Examples of such conditions include inflammatory autoimmune diseases.

This disclosure describes identification of Fexofenadine (Fex; also referred to herein as FFD) and Terfenadine (Terf; also referred to herein as TFD) as antagonists of TNF-α signaling using a library screen. We demonstrate that Fex and Terf inhibit TNF-α activity in vitro, and inhibit NF-κB nuclear translocation and activity. Our data indicate Fex and Terf act inhibit TNF-α via activation of cPLA2. We also demonstrate therapeutic effects of Fex and Terf on inflammatory diseases in animal models.

In one aspect, this disclosure provides that Fex and Terf can be used as therapeutic agents against various TNF-associated inflammatory and autoimmune diseases, including rheumatoid arthritis and inflammatory bowel disease (IBD). The method comprises administering to a subject in need of treatment, a composition comprising, or consisting essentially of, a therapeutically effective amount of Fex and/or Terf. In one embodiment, Fex and/or Terf are the only active components in the composition. In one embodiment, the subject may be suffering from an inflammatory autoimmune disease (such as IBD and RA), but not allergies, such as seasonal allergies, food allergies and the like.

We further demonstrate that, although Fex is known to be a selective antagonist of histamine receptor 1 (HR1) (also referred to herein as histamine 1 receptor 1 (H1R1), surprisingly, the anti-inflammatory effect of Fex and Terf do not depend upon HR1. Instead, Fex and Terf may exert anti-inflammatory effects by directly inhibiting activity of cytosolic phospholipase A2 (cPLA2), such as by binding to cPLA2.

As used herein, the term “treatment” means reduction in one or more symptoms or features associated with the presence of the particular condition being treated. Treatment does not necessarily mean complete remission, nor does it preclude recurrence or relapses. Thus, treatment includes ameliorating one or more symptoms associated with an indication. Treatment can be effected over a short time period, over a medium time period, or it can be a long-term treatment, for example within the context of a maintenance therapy.

The term “effective amount” in reference to therapy or “therapeutically effective amount” as used herein is the amount sufficient to achieve, in a single or multiple doses, the intended purpose of treatment. The exact amount desired or required will vary depending on the mode of administration, patient specifics and the like. Appropriate effective amounts can be determined by one of ordinary skill in the art (such as a clinician) with the benefit of the present disclosure.

Where a range of values is provided in this disclosure, it should be understood that all intervening ranges, and each intervening value, to the tenth of the unit of the lower limit of the range and any other intervening value in that stated range is encompassed within the disclosure.

The singular form in this disclosure includes the plural form and vice versa, unless indicated otherwise.

In one aspect, this disclosure provides a method of inhibiting the activity of cPLA2 in a cell (in vitro or in vivo, such as in a subject) comprising contacting the cell with Fex, Terf or a combination thereof. In one embodiment, this disclosure provides a method for inhibiting the activity of cPLA2 in a cell in a subject comprising administering to the subject in need of treatment a composition comprising, or consisting essentially of, or consisting of, an effective amount of Fex and/or Terf. The present method can be used in the treatment of any disease where cPLA2 is implicated. For example, cPLA2 inhibitors are useful for treating inflammatory diseases, autoimmune diseases, and neurodegenerative diseases, arthritis, psoriasis, cardiovascular diseases, cancer, and the like. As such, the present method can be used to treat inflammatory diseases, autoimmune diseases, and neurodegenerative diseases, arthritis, psoriasis, cardiovascular diseases and cancer. In one embodiment, the method comprises administering to a subject who is afflicted with a neurodegenerative disease, arthritis, psoriasis, cardiovascular disease or cancer, a composition comprising, or consisting essentially of, Fex and/or Terf. In one embodiment, Fex or Terf are the only active agents present in the composition. By active agents is meant an agent that is effective for treatment of the relevant disease.

Given the benefit of identification in this disclosure of the mechanism by which Fex and Terf act to exert anti-inflammatory effects, this disclosure describes a combination treatment for autoimmune inflammatory diseases in which a combination of agents can be used. For example, a combination of agents can be used, wherein the combination inhibits TNF-α activity via different mechanisms, such as by inhibiting cPLA2 activity (such as Fex and Terf) and another mechanism. Thus, in one aspect, this disclosure provides a method of treatment of autoimmune inflammatory diseases by administration to a subject in need of treatment one or more cPLA2 inhibitors and one or more other inhibitors. An example of such a combination is: i) Fex and/or Terf, and ii) one or more of Etanercept, Infliximab, Adalimumab, Certolizumab, and Golimumab (a reference to any of these drugs includes their biosimilars). The agent or agents from i) and ii) can be administered in the same composition or different compositions, at the same time or different times, by the same route of administration or different routes of administration, and over the same period of treatment or different periods of treatment. The dosage of each of i) and ii) in the method may be at a level that is effective (in case of Fex and Terf), or is considered a clinically recommended dosage (in case of agents in ii)) for the treatment of autoimmune inflammatory diseases, when the particular agent is used alone. Or the dosage of each of i) or ii) may be at a level that is lower than the dose that is effective in case of Fex and Terf, or is considered a clinically recommended dosage (in case of agents in ii)), when the particular agent is used alone.

Additionally, identifying the mechanism of Fex and Terf on TNF signaling, also allows the use of the present agents and method for treatment of those subjects afflicted with an inflammatory autoimmune disease who are refractory to treatment with current therapeutics such as Etanercept, Infliximab, Adalimumab, Certolizumab, and Golimumab, and the like. Thus, in one embodiment, this disclosure provides a method of treating an inflammatory autoimmune disease in a subject, who has not responded, or not responded to a clinically acceptable level, to treatment with Etanercept, Infliximab, Adalimumab, Certolizumab, or Golimumab, and administering to such subject a composition comprising, or consisting essentially of, Fex and/or Terf. In one embodiment, the method may comprise treating a subject having an inflammatory autoimmune disease with Etanercept, Infliximab, Adalimumab, Certolizumab, or Golimumab, evaluating if the subject is responding to the treatment, and if the subject is not responding, then treating the subject with Fex or Terf.

In one aspect, this disclosure provides compositions comprising i) Fex and/or Terf, and ii) one or more of Etanercept, Infliximab, Adalimumab, Certolizumab, and Golimumab, or biosimilars thereof. The amount of i) and ii) in the composition may be at a level that is effective (in the case of i) or considered a clinically recommended dosage for treatment of autoimmune inflammatory diseases (in the case of ii), or i) or ii) may be present at dosages lower than the effective dose (in the case of i) or clinically recommended dosage (in the case of ii) for the treatment of autoimmune inflammatory disease when the particular agent is used alone.

The term “therapeutically effective amount” as used herein in reference to a single agent is the amount sufficient to achieve, in a single or multiple doses, the intended purpose of treatment, when the agent is used alone. The term “therapeutically effective amounts” of multiple agents if it is a combination refers to the amount(s) of each agent in the combination sufficient for the combination to achieve, in a single or multiple doses, in the same or separate compositions the intended purpose of treatment. For example, this disclosure provides the administration of therapeutically effective amounts of agents as a combination (whether as a single composition or separate compositions) that will alleviating one or more symptoms of an inflammatory autoimmune disease. The term “sub-therapeutic amount” as used herein means an amount of an agent that by itself is not considered suitable for treatment of a condition. However, as described herein a sub-therapeutic amount of a first agent (Fexofenadine or Terfenadine) may be combined with a therapeutic or sub-therapeutic amount of another agent (e.g., a TNF-α inhibitor that does not act via cPLA2 via HR1). Similarly, therapeutic amount of Fexofenadine or Terfenadine can be combined with a therapeutic or sub-therapeutic amount of a TNF-α inhibitor that does not act via cPLA2. Examples of TNF-α inhibitors that do no act via cPLA2 include Etanercept, Infliximab, Adalimumab, Certolizumab, and Golimumab, or biosimilars thereof.

In an embodiment, for combination treatment using Fex (or Terf) with Etanercept, Infliximab, Adalimumab, Certolizumab, or Golimumab, the dose of each active component of the combination (e.g., Fex and one of Etanercept, Infliximab, Adalimumab, Certolizumab, and Golimumab) may be reduced to ¾^{th}, ½ or ¼^{th} of the dose when given alone. In an embodiment, the dose of each active component in the combination may be reduced to 1/10^{th} of the dose when given alone. In an embodiment, the dose of each active component in a combination may be from 90% to 10% of the dose when given alone.

In a specific embodiment, Fex or Terf is administered in combination with Etanercept, Infliximab, Adalimumab, Certolizumab, or Golimumab for the treatment of inflammatory autoimmune diseases, such as IBD and rheumatoid arthritis (RA). In an embodiment, Fex or Terf is administered in combination with Infliximab or Etanercept.

Generally, the therapeutic dose of Fex and Terf is in the range of 1 μg to 1,000 mg per dose. In an embodiment, the amount may be 1 mg to 1,000 mg per dose. For example, typical doses may be in the range of 10 mg to 800 mg, which may be given once or twice a day. As an example, Fexofenadine hydrochloride 60 mg twice daily or 180 mg once daily can be used. A dose of 60 mg once daily can also be used. It is generally considered that for younger subjects, such as children 6 to 11 Years, the recommended dose of Fexofenadine hydrochloride is 30 mg twice daily. In one example, subjects from 6 months to 2 years can be given 15 mg orally 2 times a day; 2 years to 11 years can be given 30 mg orally 2 times a day; and 12 years and older can be given 180 mg orally once a day or 60 mg orally 2 times a day. In an embodiment, suitable dose of Fex may be in the range of 0.001 to 10 mg/kg body weight. In an embodiment the dose of Fex may be 0.001 to 1 mg/kg body weight. In an embodiment, the dose may be in the range of 0.003 to 0.76 mg/kg in human. As an example, for a person with 60 kg bodyweight, the dose may be in the range of 1 to 50 mg. Given the benefit of this disclosure, one skilled in the art can determine appropriate dosage for present methods without undue experimentation. While data is provided in this disclosure for animal models using certain doses of Fex or Terf and for the combination of Fex with Etanercept, Infliximab, Adalimumab, Certolizumab, and Golimumab, based on the data and knowledge in the art, appropriate doses for human use may be determined by routine methods. An example of conversion guidance from animal doses to human doses can be found in Nair et al., J Basic Clin Pharma 2016; 7:27-31, incorporated herein by reference.

Suitable doses of TNF-α inhibitors, such as Etanercept, Infliximab, Adalimumab, Certolizumab, and Golimumab, are well known in the art. For example, a clinical starting dosage of Enbrel in RA patients is 50mg (for a 60 kg individual). In a combination treatment, the dose of Enbrel may be reduced to 5 mg in combination with Fex.

In an embodiment, Fex or Terf may also be used in combination with another HR1 inhibitor, such as, for example for treatment of RA or IBD.

Administration of present compositions, separately or as a single composition, as described herein can be carried out using any suitable route of administration known in the art. For example, the compositions may be administered via intravenous, intramuscular, intraperitoneal, intracerobrospinal, subcutaneous, intra-articular, intrasynovial, oral, topical, or inhalation routes. The compositions may be administered parenterally or enterically. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be clinically needed and/or therapeutically indicated. The treatment can be carried on as long as clinically needed and/or therapeutically indicated.

The agents of the present disclosure, or pharmaceutically acceptable salts thereof (such as, but not limited to, hydrochloride), can be provided in pharmaceutical compositions for administration by combining them with any suitable pharmaceutically acceptable carriers, excipients and/or stabilizers. Examples of pharmaceutically acceptable carriers, excipients and stabilizer can be found in Remington: The Science and Practice of Pharmacy (2005) 21st Edition, Philadelphia, Pa. Lippincott Williams & Wilkins. For example, suitable carriers include excipients, or stabilizers which are nontoxic to recipients at the dosages and concentrations employed, and include buffers such as acetate, Tris, phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; and/or non-ionic surfactants such as Tween or polyethylene glycol (PEG). The pharmaceutical compositions may comprise other therapeutic agents.

The subject treated with the compositions and methods of this disclosure can be a human subject or a non-human animal. The subject can be of any gender or age.

In an aspect, this disclosure provides a synergistic composition comprising Fexofenadine and/or Terfenadine, and one of Etanercept, Infliximab, Adalimumab, Certolizumab, or Golimumab. In an embodiment, the combination comprises Fexofenadine (cPLA2 affecting component) and one of Etanercept, Infliximab, Adalimumab, Certolizumab, or Golimumab (non-cPLA2 affecting component), wherein either the Fexofenadine or the one of Etanercept, Infliximab, Adalimumab, Certolizumab, or Golimumab is present at a subtherapeutic dose with respect to treatment of inflammatory autoimmune diseases. In an embodiment, both the cPLA2 affecting component, and the non-CPLA2 component are present at sub-therapeutic doses. For example, both fexofenadine and one of Etanercept, Infliximab, Adalimumab, Certolizumab, or Golimumab are present at sub-therapeutic dose. The combination may be present in a pharmaceutically acceptable carrier and may further comprise routine excipients and the like.

This disclosure also provides kits or packages comprising Fex (or Ted) and optionally one or more of Etanercept, Infliximab, Adalimumab, Certolizumab, Golimumab. The Fex and one of Etanercept, Infliximab, Adalimumab, Certolizumab, Golimumab may be present in the same composition or different compositions. The Fex and one of Etanercept, Infliximab, Adalimumab, Certolizumab, Golimumab may be provided in a lyophilized, freeze-dried form for preparing as a liquid formulation at the time of administration. For example, Fex and one of Etanercept, Infliximab, Adalimumab, Certolizumab, Golimumab may be present in separate packaging (such as blister packaging) in a kit. A carrier solution or suspension, such as physiological saline or some other salt of buffer solution, may be provided for each together or separately. Instructions may also be included in the kit with guidance of preparation of the medicaments and/or dosage, delivery etc. In an embodiment, the amounts of each component provided for a dose is less than a therapeutic dose. For example, the dose of each can be less (such as from 90% to 10%) than the dose that can be used for treatment of an inflammatory autoimmune disease if the component is used by itself, without the other component.

In an embodiment, this disclosure provides a method of treating an inflammatory autoimmune disease comprising administering to a subject in need of treatment, a composition comprising or consisting essentially of Fexofenadine and/or Terfenadine. In an embodiment, this disclosure provides a method for treating an inflammatory autoimmune disease comprising administering to a subject in need of treatment i) Fexofenadine and/or Terfenadine, and ii) another TNF-α inhibitor, wherein i) and ii) can be administered in the same composition or different compositions, same time or different times, by same route of administration or different routes of administration, over the same length of treatment or different lengths of treatment. The other TNF-α inhibitor can be Etanercept, Infliximab, Adalimumab, Certolizumab, or Golimumab. The dose of i) can be less than the dose used if i) is used without ii), and/or the dose of ii) can be less than the dose used if ii) is used without i). For example, the doses of one or each of i) and ii) in the combination can be from 90% to 10% of the doses when used without the other. The method may be used for the treatment of any inflammatory autoimmune disease. For example, the method may be used for the treatment of inflammatory bowel disease or rheumatoid arthritis.

In an embodiment, this disclosure provides a method for inhibiting cPLA2 in a cell in a subject comprising administering to a subject in need of treatment a composition comprising or consisting essentially of Fexofenadine and/or Terfenadine. For example, the disclosure provides a method of treating a cPLA2 associated disorder comprising administering to a subject afflicted with the disorder, a composition comprising or consisting essentially of Fexofenadine and/or Terfenadine. The cPLA2 associated disorder may be arthritis, psoriasis, cardiovascular disease, neurodegenerative disease, or cancer. The neurodegenerative disease may be Alzheimer's disease.

In an embodiment, this disclosure provides a method of treating a subject having an inflammatory autoimmune disease who has not responded to treatment with one of Etanercept, Infliximab, Adalimumab, Certolizumab, Golimumab, comprising administering to said subject a composition comprising or consisting essentially of Fexofenadine or Terfenadine. The inflammatory autoimmune disease may be IBD or RA. In an embodiment, the method can comprise treatment of an individual who has not responded to treatment with Etanercept, Infliximab, Adalimumab, Certolizumab, Golimumab, wherein the treatment comprises continuing administration of Etanercept, Infliximab, Adalimumab, Certolizumab, Golimumab, and further administering Fex or Terf at therapeutic or sub-therapeutic doses to the individual. The dosage of Etanercept, Infliximab, Adalimumab, Certolizumab, and Golimumab can be reduced.

In an embodiment, this disclosure provides a method of treating a subject having an inflammatory autoimmune disease a subtherapeutic dose of Fexofenadine or Terfenadine, and a subtherapeutic dose of one of Etanercept, Infliximab, Adalimumab, Certolizumab, and Golimumab. A subtherapeutic dose is one where the dose alone does not or is not expected to provide a treatment for the indication. For example, a subtherapeutic dose can be from 90% to 10% of the effective dose when the therapeutic is used alone. When a combination treatment comprising administering to a subject a dose of fexofenadine (or Terf), and one of Etanercept, Infliximab, Adalimumab, Certolizumab, or Golimumab, and either one or both components are present at a dose which if given alone is not expected to treat the disease, then the composition is considered to be a synergistic combination of the two components.

The following examples are provided as illustrative examples and are not intended to be restrictive in any way.

### EXAMPLE 1

This example demonstrates the identification of inhibitors of TNF-α signaling. Serial screenings of a library composed of FDA-approved drugs resulted in identification of fexofenadine as an inhibitor of TNF-α signaling. Fexofenadine potently inhibited TNF/nuclear factor kappa-light-chain-enhancer of activated B cells (NF-κB ) signaling in vitro and in vivo, and ameliorated disease symptoms in inflammatory arthritis models. cPLA2 was isolated as a novel target of fexofenadine. Fexofenadine blocked TNF-stimulated cPLA2 activity and arachidonic acid production through binding to catalytic domain 2 of cPLA2 and inhibition of its phosphorylation on Ser-505. Further, deletion of cPLA2 abolished fexofenadine's anti-TNF activity. Collectively, these findings provide new insights into the understanding of fexofenadine action and underlying mechanisms and also provide new therapeutic interventions for various TNF-α and cPLA2-associated pathologies and conditions, particularly inflammatory rheumatic diseases.

We adopted a strategic approach involving repurposing clinically approved drugs. A drug library composed of FDA-approved drugs was screened both in vitro and in vivo by use of TNF-α/NF-βB reporter constructs and mice, which led to the identification of terfenadine and its active metabolite fexofenadine as inhibitors of TNF-α signaling.

We identified cytosolic phospholipase A2 (cPLA2) as a novel target of fexofenadine. The major function of cPLA2 is to promote phospholipid hydrolysis-mediated production of arachidonic acid (AA); AA activates NF-κB and is involved in the pathogeneses of various conditions, including inflammatory and auto-immune diseases. In the present example, we provide data demonstrating that fexofenadine acts as the inhibitor of TNF/NF-κB signaling and is therapeutic against inflammatory arthritis. Additionally, we also provide evidences revealing that this drug bound to cPLA2 and inhibited its enzymatic activity, which is required for its inhibition of TNF-α signaling.

Results

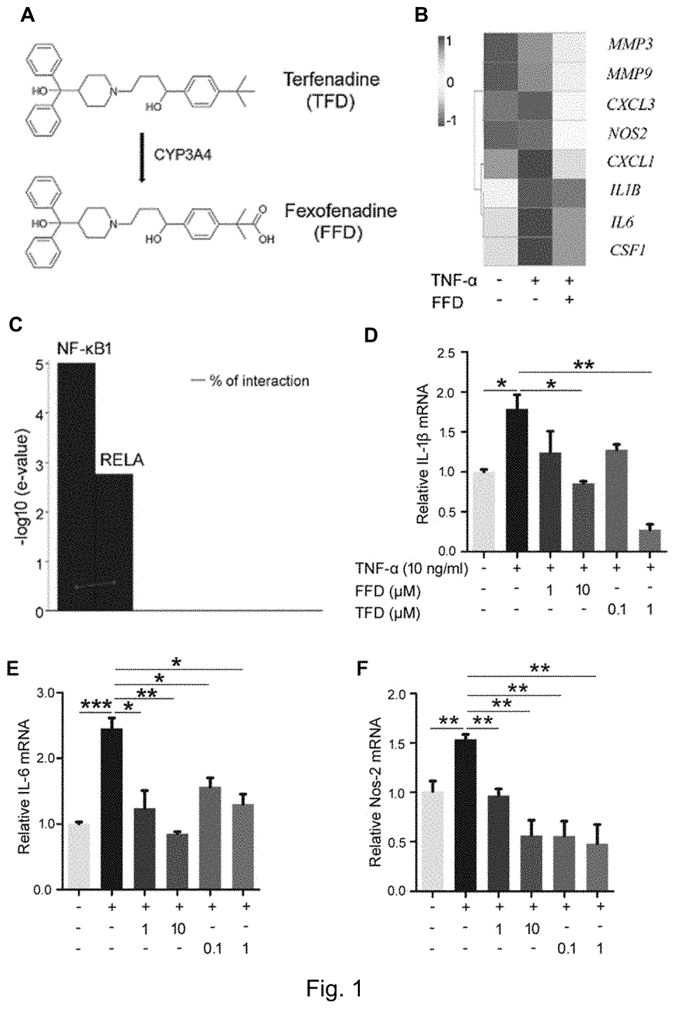

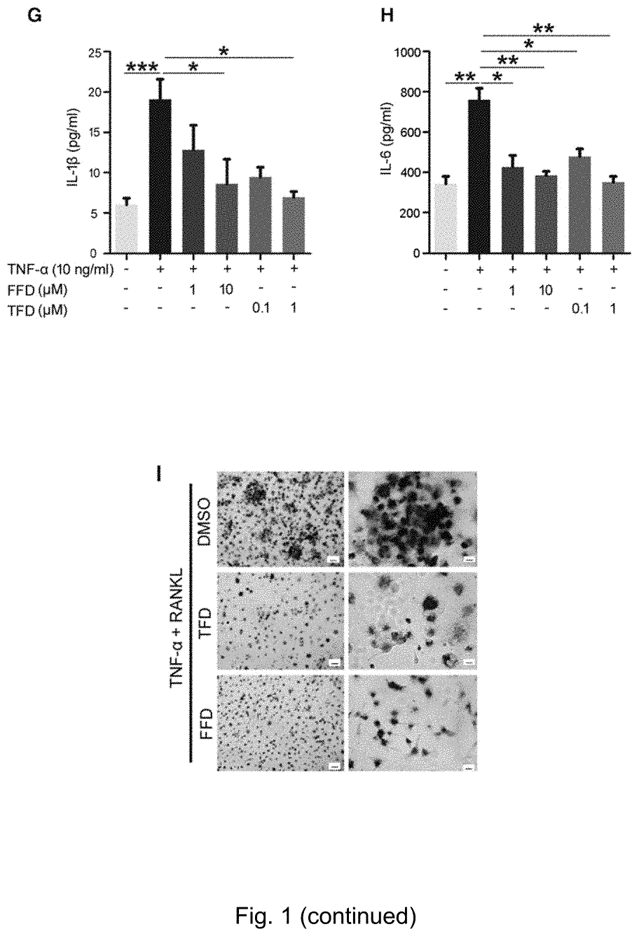

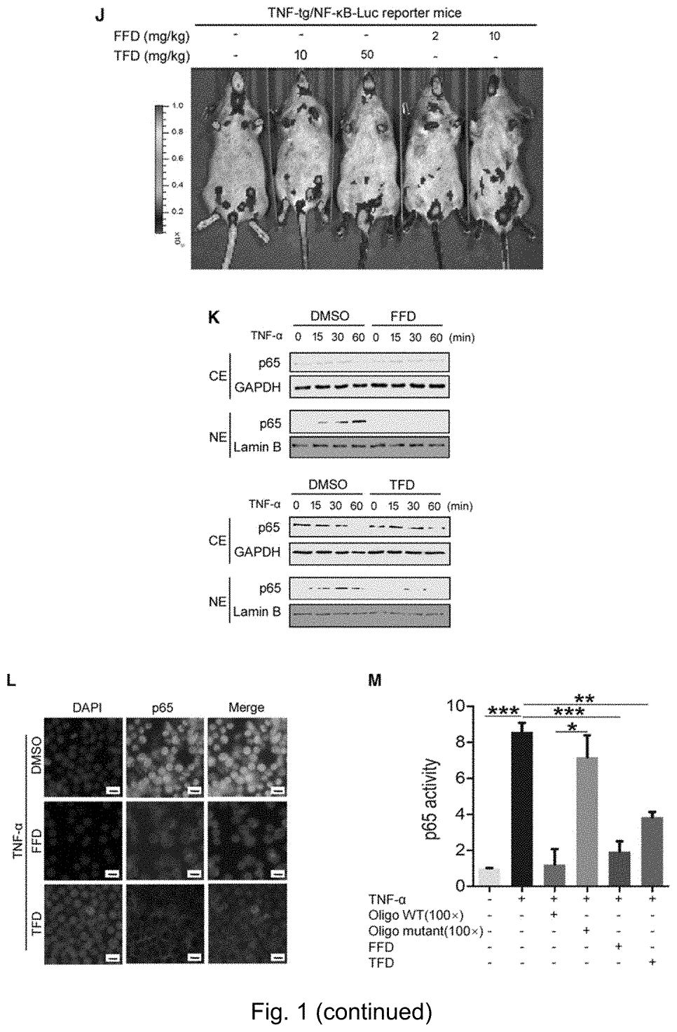

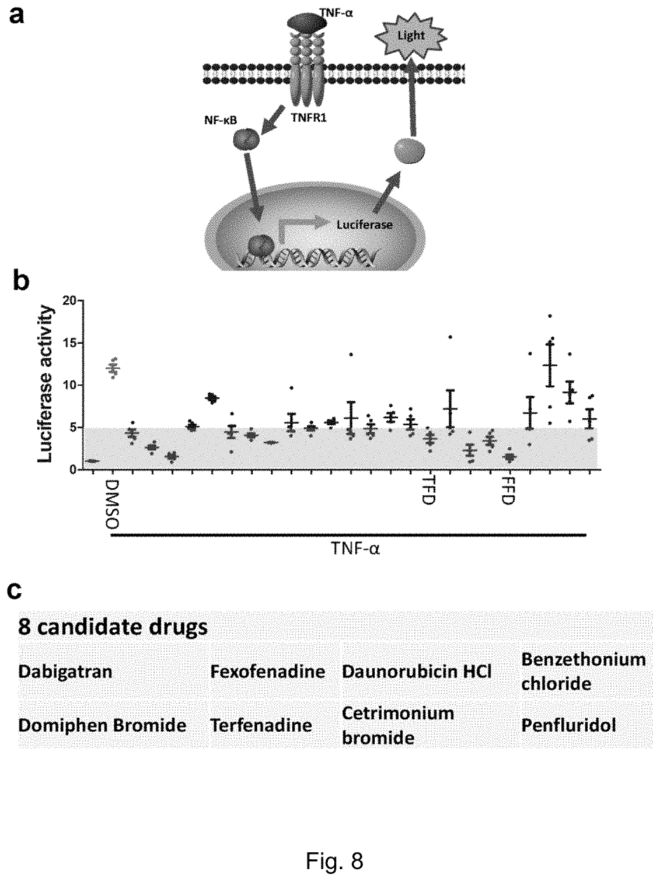

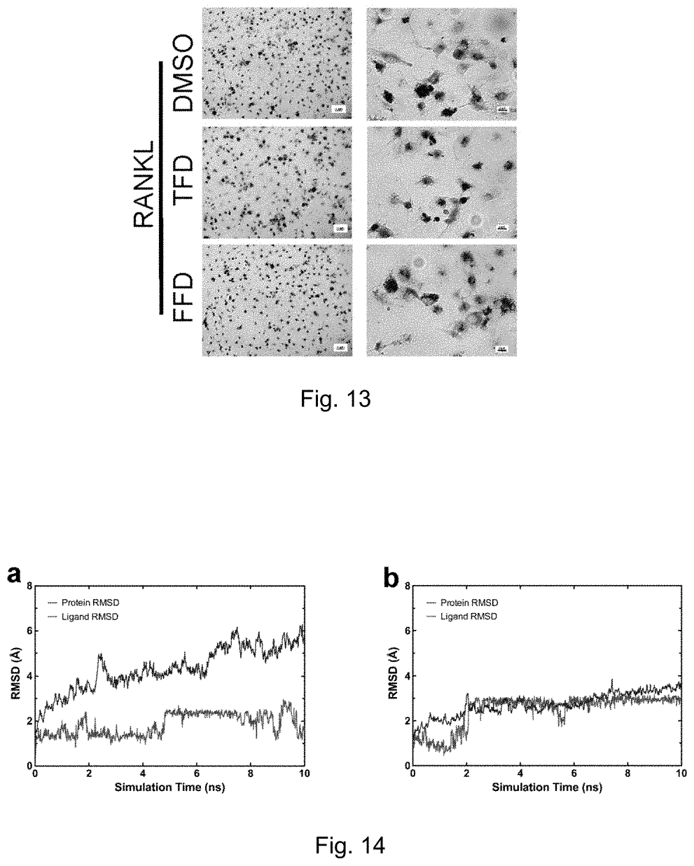

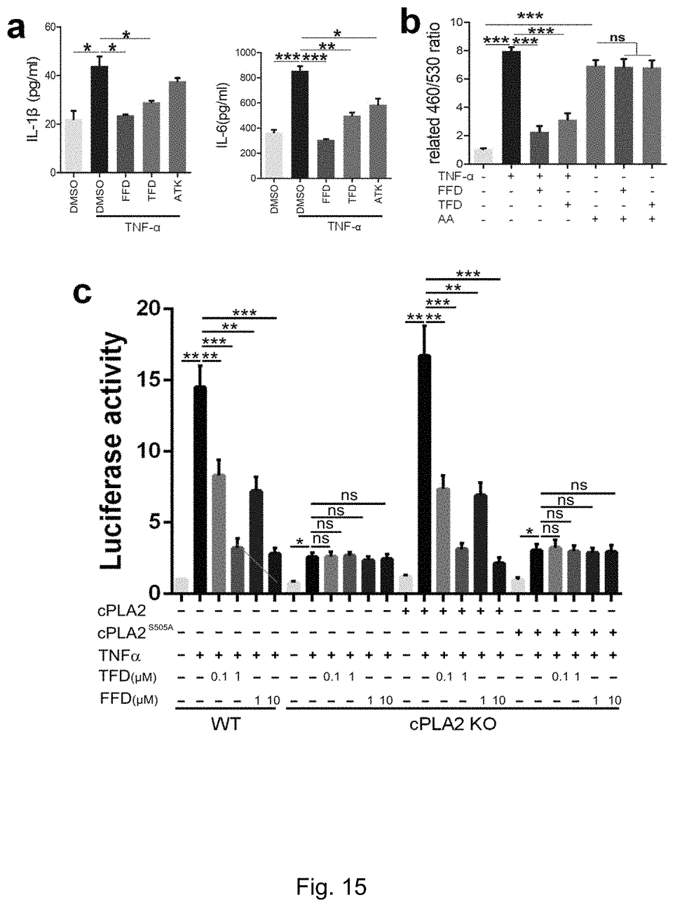

Fexofenadine is identified as an antagonist of TNF-α and inhibits TNF-α signaling and activity. To isolate the small molecule drugs that inhibit canonical TNF-α/NF-κB signaling pathway, a drug library containing 1046 FDA-approved drugs was initially screened using a NF-κB-bla THP-1 cell line in which a NF-κB beta-lactamase reporter gene was stably integrated. Twenty-four drugs that potently inhibited TNF-α/NF-κB activation of beta-lactamase were identified after three independent implementations of this screening scheme (FIGS. 13a-b). These 24 isolates were subjected to a second round screen using RAW 264.7 macro-phages transiently transfected with an NF-κB luciferase reporter gene. Under such conditions, only the most potent anti-TNF-α/NF-κB signaling drugs are positively screened. Eight drugs among the 24 candidates originally isolated were selected (online supplementary FIGS. 8a-b). In order to identify the drugs that retain anti-TNF-α/NF-κB activity in vivo, we performed a third round screen with NF-κB-Luc reporter mice. We first crossed TNF-α transgenic (TNF-tg) to NF-κB-Luc reporter mice to generate TNF-tg:NF-κB-Luc double mutant mice. Overexpression of TNF-α effectively activated NF-κB luciferase in vivo. In Vivo Imaging System (IVIS) was implemented for whole animal bioluminescence imaging following intraperitoneal injection of eight selected drugs into TNF-tg:NF-κB-Luc double mutant mice. Five drugs, including terfenadine and its active metabolite fexofenadine, were shown to effectively inhibit TNF-tg:NF-κB activated luciferase in vivo (FIG. 15). Among these five drugs, three, including one anticancer drug, are known to have severe side-effects and are not suitable for treating chronic inflammatory diseases, such as rheumatoid arthritis, we accordingly selected fexofenadine and terfenadine (serving as a comparison with fexofenadine) for further analyses (FIG. 1A).

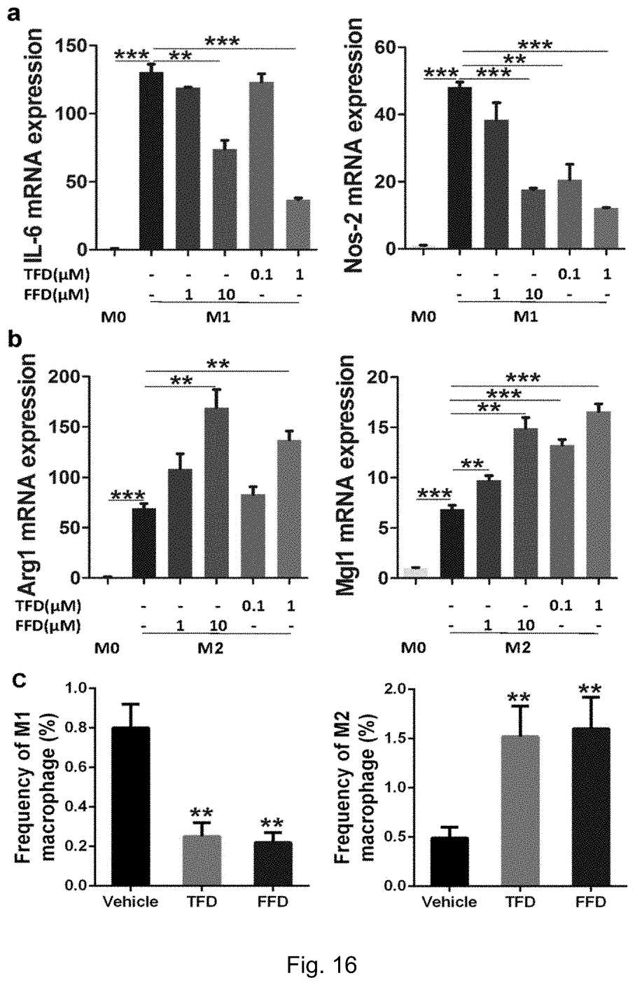

We first examined the inhibition of fexofenadine on TNF-α-activated NF-κB pathway and downstream genes through RNA-seq with bone marrow-derived macrophages (BMDMs) (FIGS. 1B-C, FIG. 16). Nearly, all TNF-α-induced genes, especially genes encoding inflammatory cytokines, such as IL-10, IL-6, were clearly downregulated by fexofenadine and terfenadine. The lists of TNF-α inducible genes that were inhibited by fexofenadine were used for transcription factor enrichment analysis with TFactS, which led to the isolation of NF-κB p105 and RelA p65 as transcription factors significantly regulated by fexofenadine (FIG. 1C).

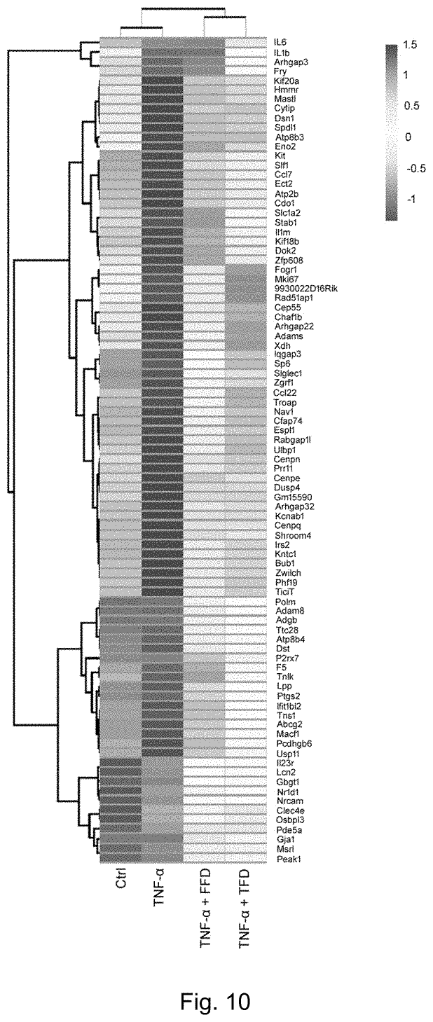

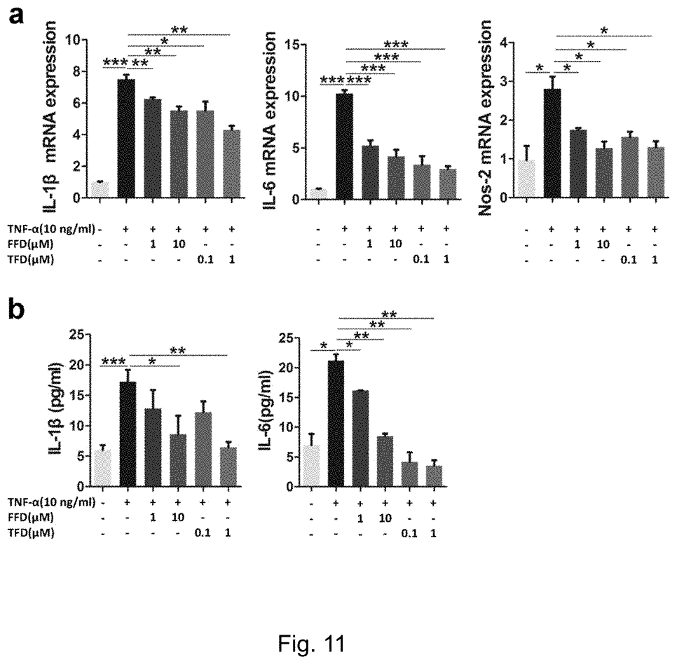

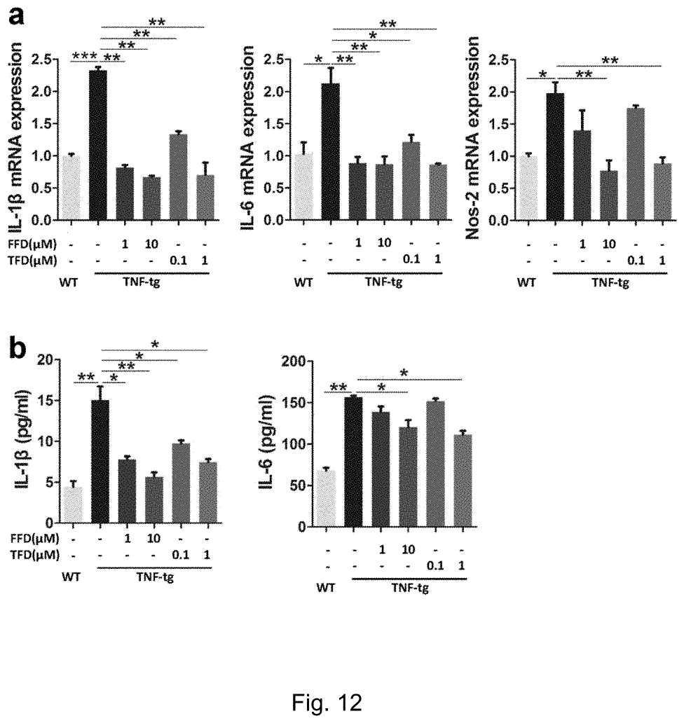

In order to further validate the anti-TNF-α activity of fexofenadine, we next selected a couple of well-known TNF-α down-stream inflammatory mediators for further assays. Quantitative real-time PCR revealed that both fexofenadine and terfenadine dose-dependently inhibited TNF-α-induced mRNA expressions of IL-1β, IL-6 and Nos-2 in BMDMs (FIGS. 1D-F). Addition-ally, ELISA demonstrated that these two drugs abolished TNF-α induced releases of IL-1β and IL-6 in a dose-dependent manner (FIGS. 1G-H). Similar anti-TNF activity of fexofenadine and terfenadine was also observed in RAW264.7 cells (FIGS. 11a-b) and BMDMs isolated from TNF-tg mice (FIGS. 12a-b). TNF-α is known to enhance RANKL-stimulated osteoclastogenesis. Both fexofenadine and terfenadine markedly inhibited TNF-α-mediated osteoclastogenesis in BMDMs (FIG. 11), but not RANKL-induced osteoclastogenesis (FIG. 13). Moreover, in vivo dose-dependent inhibition of the TNF-α/NF-κB pathway by fexofenadine and terfenadine was also revealed by use of TNF-tg/NF-κB-Luc reporter double mutant mice (FIG. 1J). Additionally, the TNF-α-induced nuclear translocation and DNA-binding activity of p65 were also inhibited by fexofenadine-dine and terfenadine (FIGS. 1K-M).

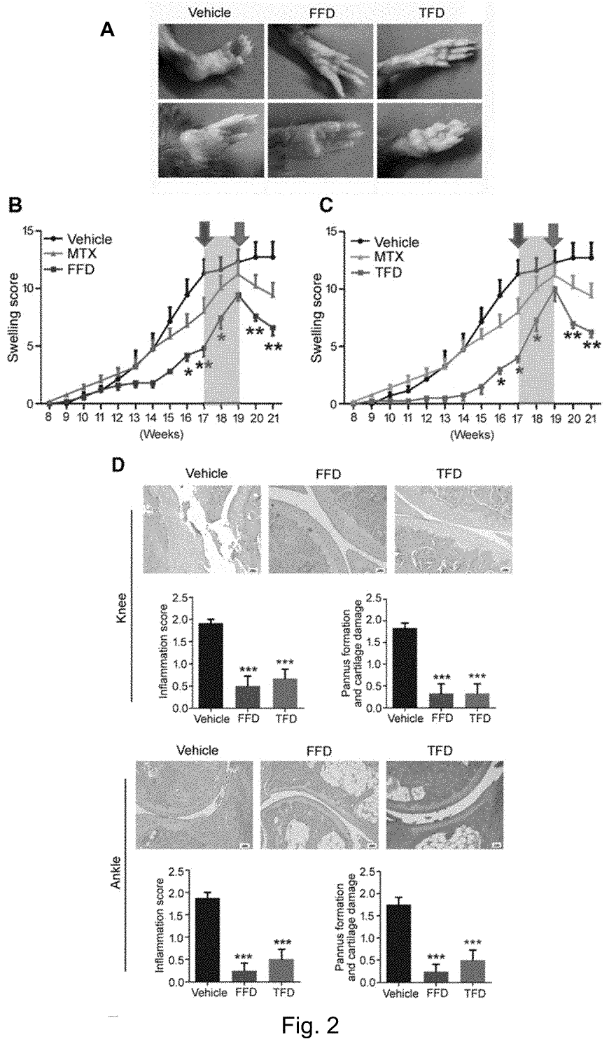

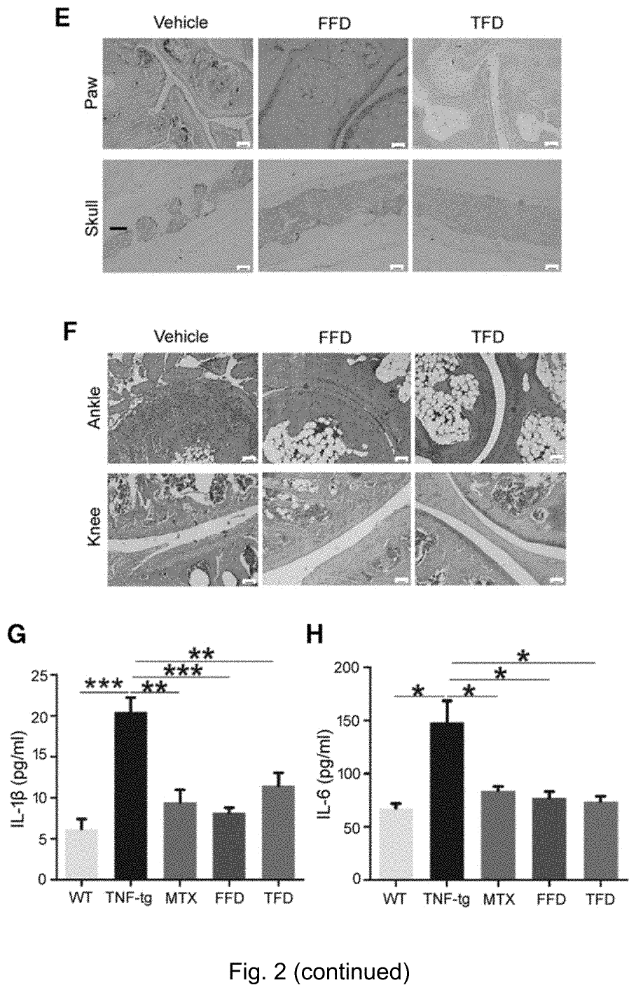

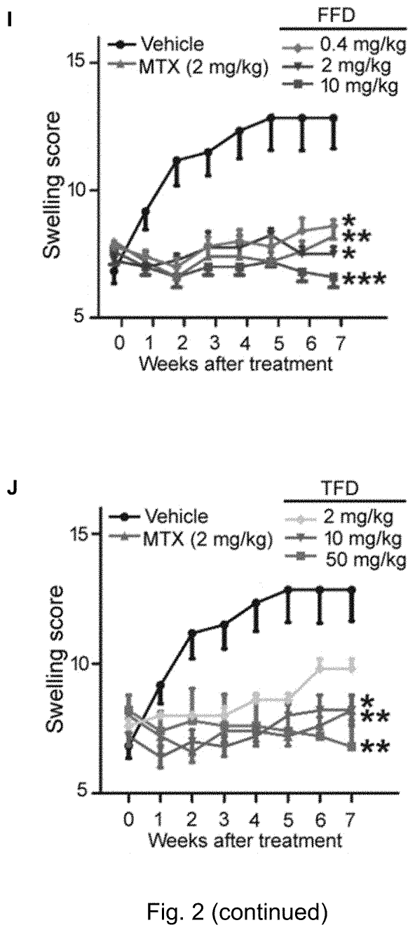

Fexofenadine prevents the spontaneous development of inflammatory arthritis in TNF-tg mice. TNF-tg mice are known to develop an inflammatory arthritis phenotype spontaneously when mice reach 12-16 weeks old. Next, we sought to examine the effects of applying fexofenadine to TNF-Tg mice. First, 8-week-old TNF-tg mice were treated daily with fexofenadine, terfenadine or methotrexate (MTX, serving as a positive control) by oral delivery before the onset of the inflammatory arthritis phenotype. Both fexofenadine and terfenadine treatment resulted in reduction of all visual symptomatic signs (FIG. 2A) and significant reduction of clinical scores of arthritis; fexofenadine was proven to be more effective than MTX, the current clinically used small molecule drug for treating rheumatoid arthritis (FIGS. 2B-C). In order to observe the response of inflammatory arthritis progression to fexofenadine and terfenadine, we stopped treatment at the 17-week-time point and resumed treatment at the 19-week-time point. Cessation of treatment led to an abrupt increase of the arthritis clinical scores. Once the treatment resumed, there was an immediate reduction in swelling score, indicating that the inflammatory arthritis induced by TNF-α overexpression responds well to both fexofenadine and terfenadine (FIGS. 2B-C). H&E staining of ankle and knee tissues confirmed the inhibition of inflammatory degeneration (FIG. 2D). TRAP staining of paw and skull showed a preventative effect of treatment on osteoclast differentiation (FIG. 2E). In addition, the drugs reduced cartilage loss, as revealed by Safranin 0 staining of ankle and knee (FIG. 2F). We also measured the serum levels of IL-1β and IL-6 and found that the levels of these inflammatory cytokines were significantly reduced in fexofenadine-treated and terfenadine-treated groups compared with the control group (FIGS. 2G-H).

To determine drug's therapeutic effects, we started treatment when the TNF-tg mice reached an average score of approximately eight points. Both fexofenadine and terfenadine showed effective therapeutic effects in a dose-dependent manner (FIGS. 2I-J). Taken together, data from TNF-tg mice indicate that fexofenadine and terfenadine exert their anti-inflammatory and therapeutic effects through the inhibition of TNF-α activity in vivo.

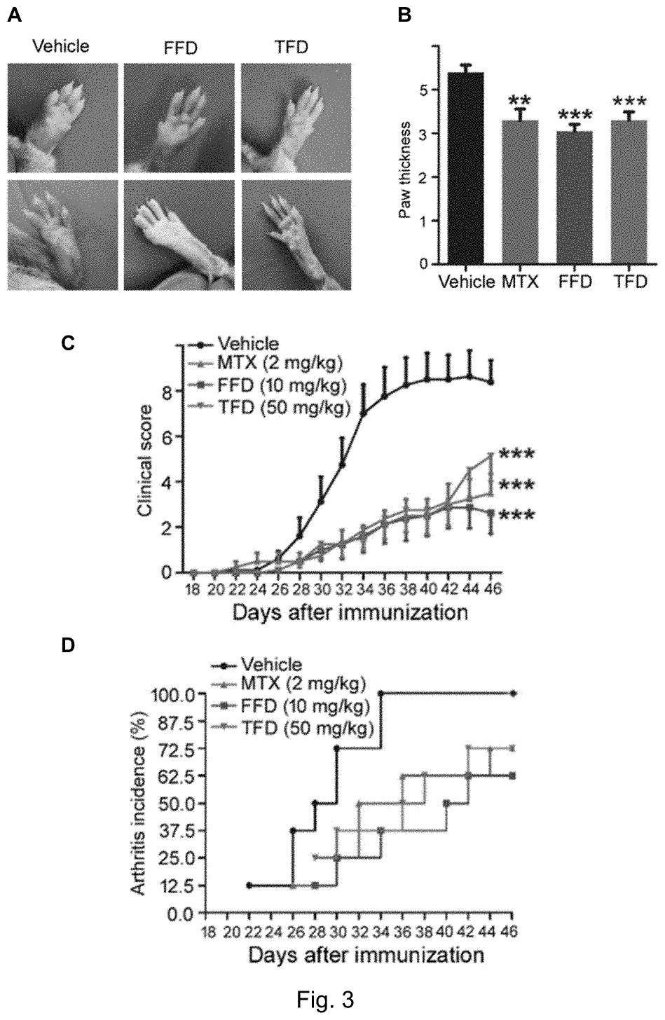

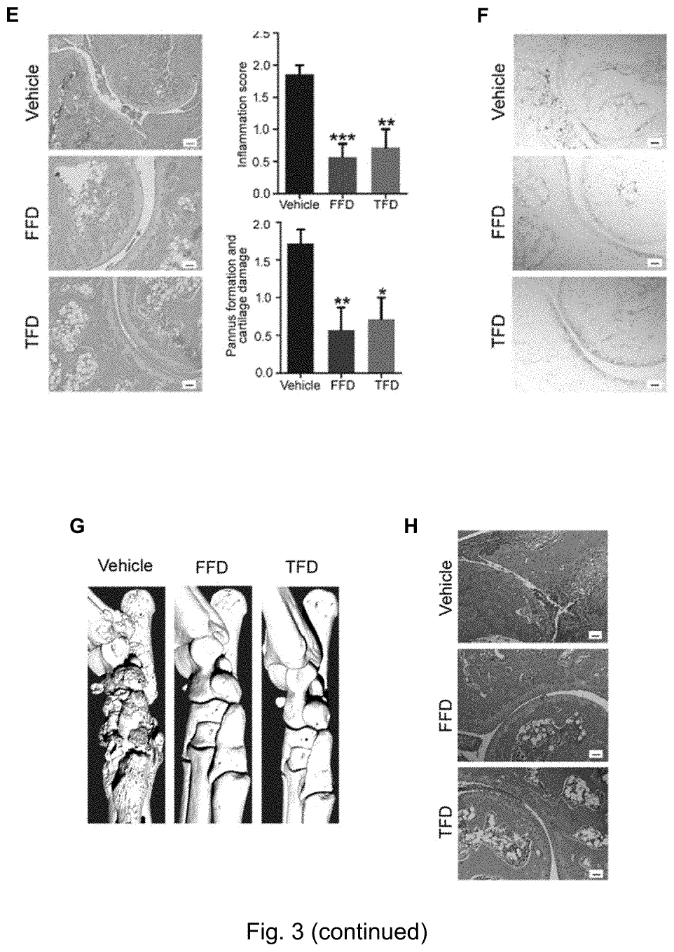

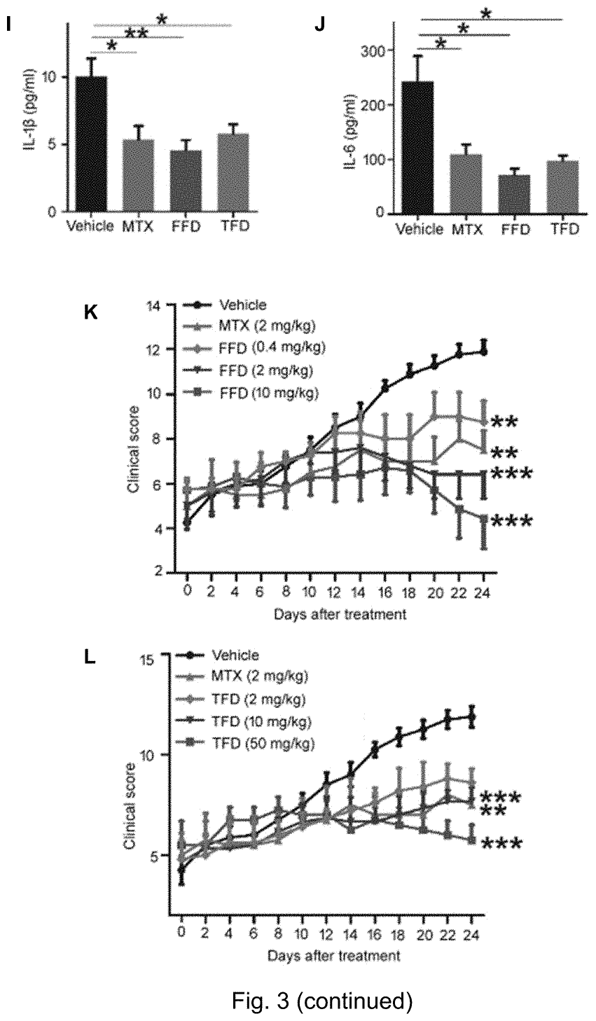

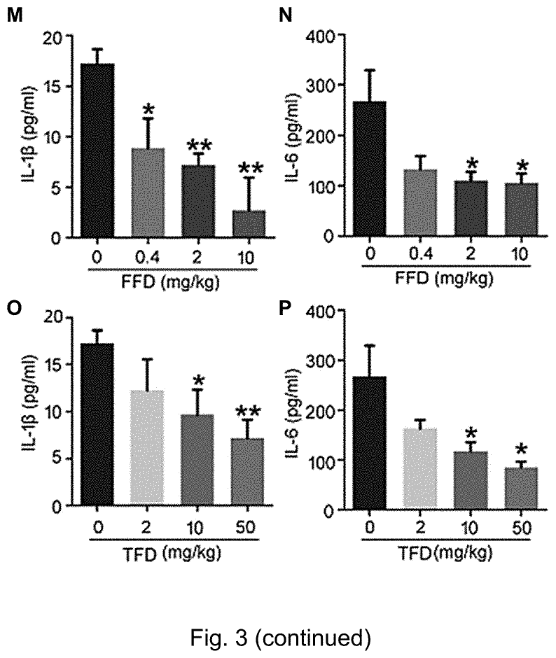

Fexofenadine prevents the onset and progression of collagen-induced arthritis. To advance understanding of the preventive and therapeutic impact of fexofenadine on inflammatory arthritis in vivo, we utilized another mouse model of rheumatoid arthritis: collagen-induced arthritis (CIA), which has both immunological and pathological features with rheumatoid arthritis. The CIA model was established with 8-week-old male DBA/1J mice. We first started the treatment with fexofenadine, terfenadine, MTX or vehicle by oral delivery at 18 days after immunization for examining their preventive effects. Severe inflammation and increased thickness in the ankles and paws were observed in the vehicle group compared with intervention groups (FIGS. 3A-B). Analogously, fexofenadine and terfenadine could not only delay the onset of disease but also significantly decrease arthritis clinical scores and incidence (FIGS. 3C-D). Histological analysis revealed less inflammation in treatment groups as compared with control group (FIG. 3E). Fewer osteoclasts and less bone destruction were detected in the treated groups, as revealed by TRAP staining and microCT images (FIGS. 3F-G). Additionally, fexofenadine and terfenadine also prevented the loss of cartilage (FIG. 3H), and significantly reduced the serum levels of IL-1β and IL-6 (FIGS. 3I-J).

To determine drug's therapeutic effects, we initiated treatment when the CIA model mice displayed a clinical score of ˜5 points of a maximum 16 points per animal. Both fexofenadine and terfenadine dose-dependently ameliorated disease scores (FIGS. 3K-L). Meanwhile, the serum levels of inflammatory cytokines IL-1β and IL-6 were significantly decreased in the treatment groups versus vehicle (FIGS. 3M-P). Collectively, these data indicate that fexofenadine has both preventive and therapeutic effects in a well-accepted preclinical animal model for testing anti-rheumatoid arthritis (RA) drugs.

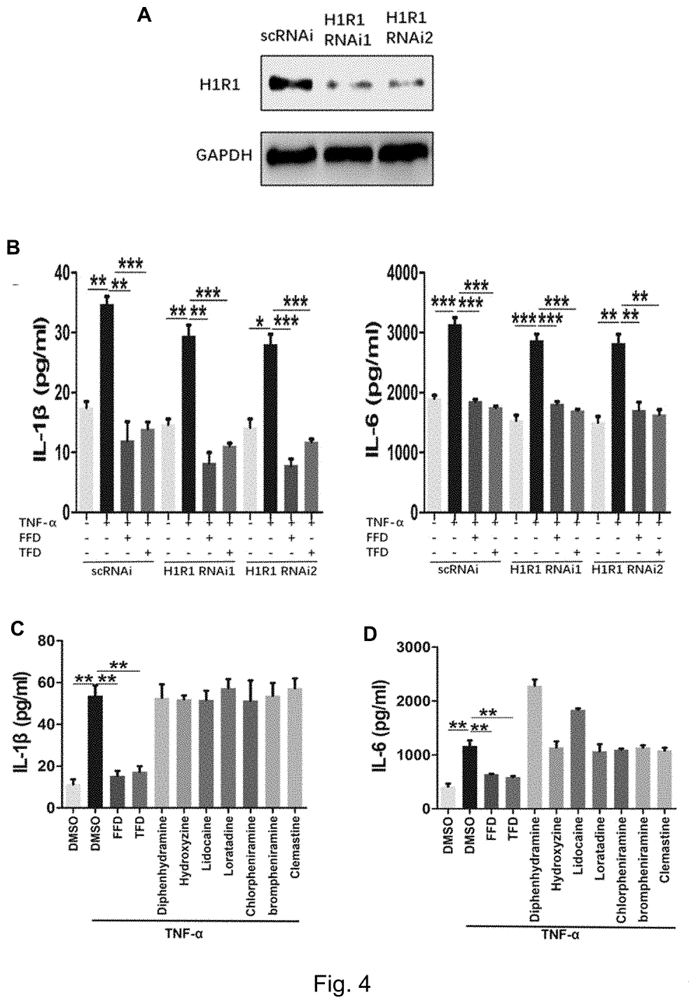

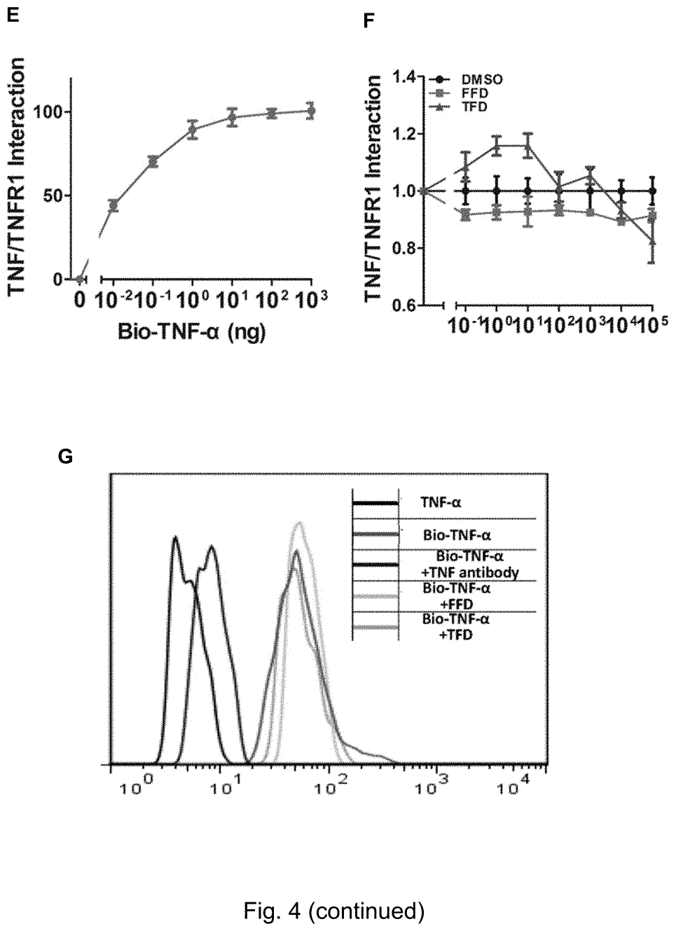

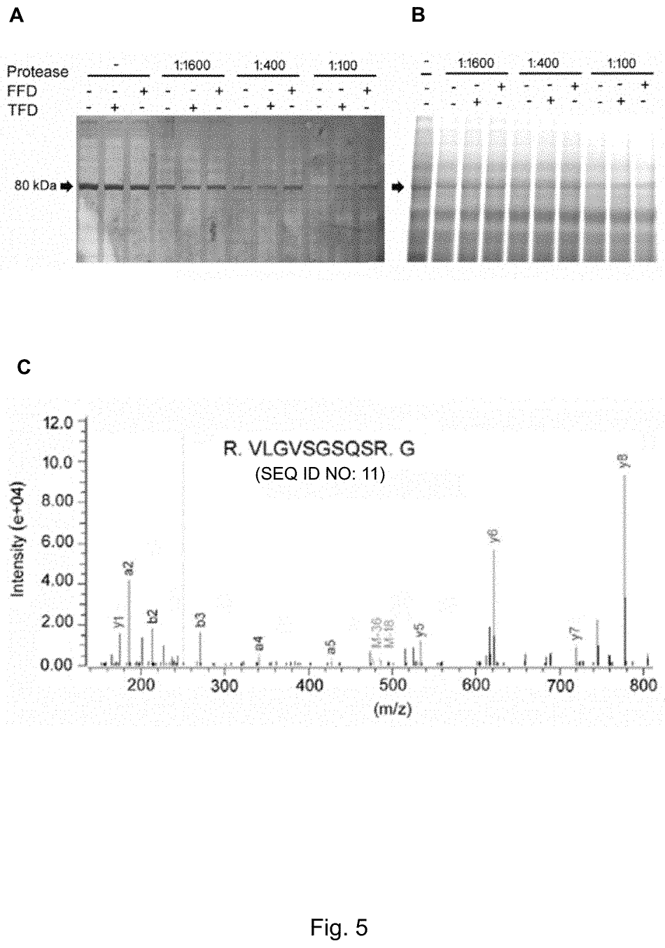

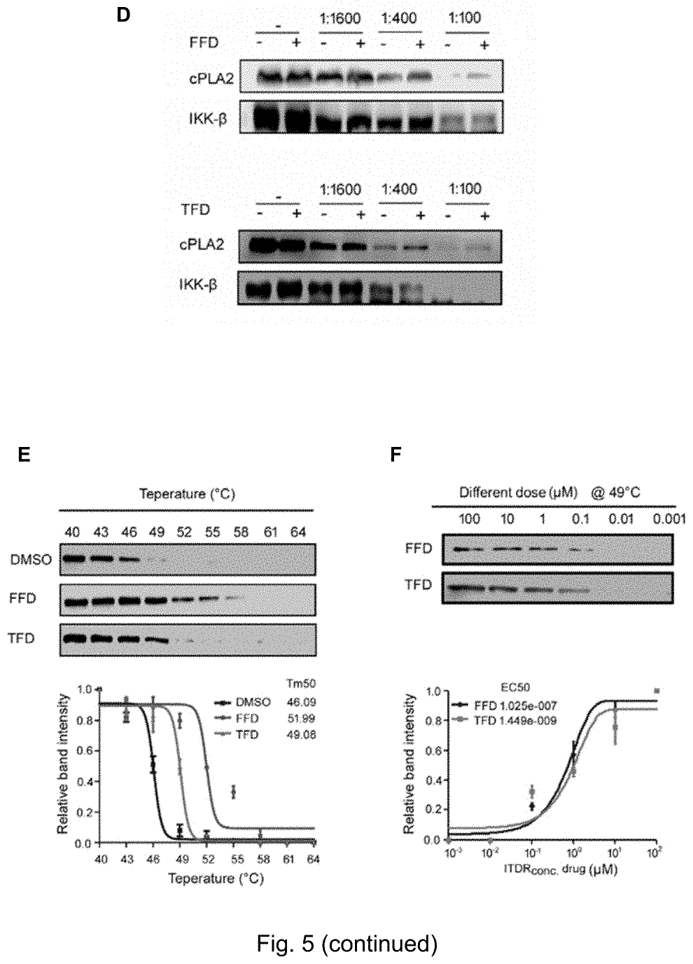

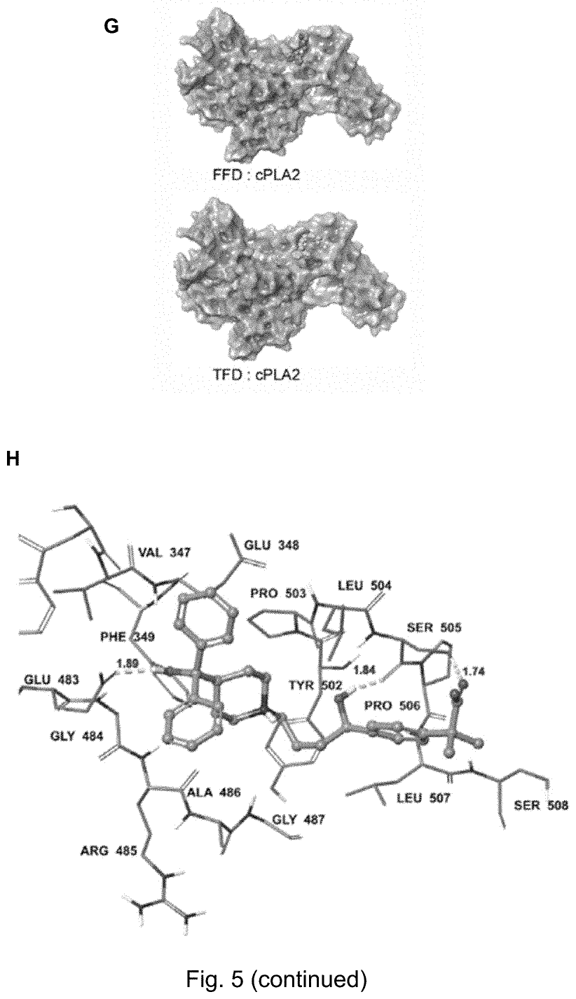

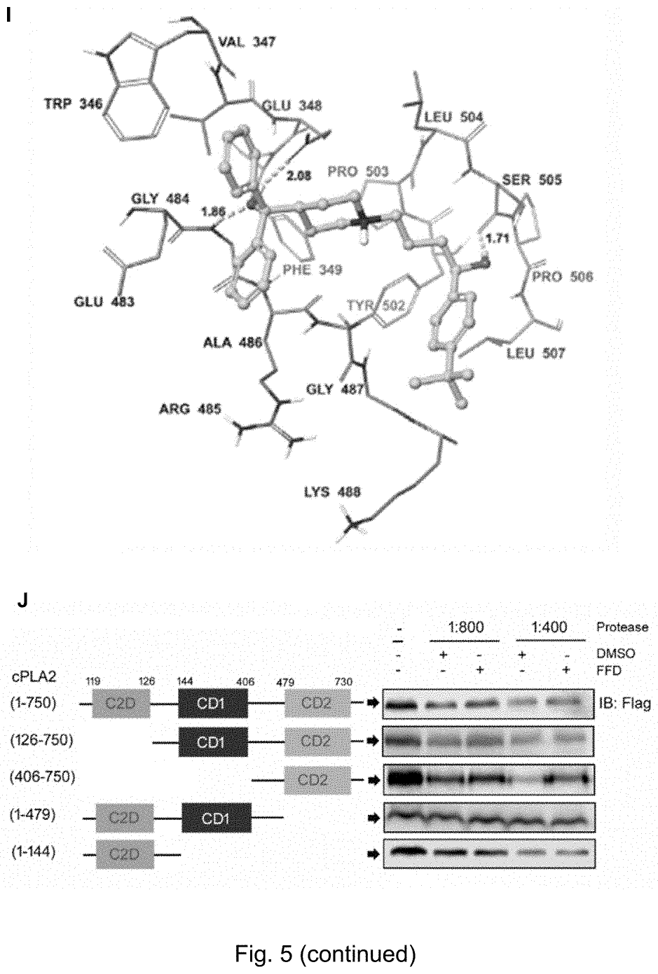

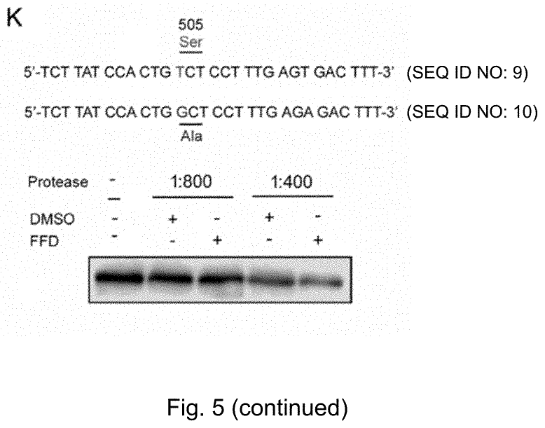

We demonstrate that cPLA2 is a novel target of fexofenadine. The antihistaminic activity of fexofenadine and terfenadine are known to be mediated by targeting to their selective histamine H1 receptor 1 (H1R1). We next sought to determine whether the anti-TNF activity of fexofenadine and terfenadine depends on their known target H1R1. We thus suppressed the expression of H1R1 using its specific siRNAs in RAW264.7 cells, and found, unexpectedly, that suppression of H1R1 did not affect the inhibition of fexofenadine and terfenadine on TNF-induced cytokine release (FIGS. 4A, B). In addition, seven additional accepted H1R1 antagonists did not exhibit anti-TNF activity, with some even associated with increased TNF-induced IL-6 release (FIGS. 4C, D). Collectively, these results indicate that anti-TNF activity of fexofenadine is H1R1 independent. Current clinically employed TNF inhibitors, such as etanercept (Enbrel), infliximab (Remicade) and adalimumab (Humira), exert their anti-TNF activity through disturbing the binding of TNF to its receptor tumor necrosis factor receptor 1 (TNFR1). Therefore, we next examined whether fexofenadine affected the interactions between TNF and TNFR1, leading to its anti-TNF activity. Surprisingly, both solid phase binding and flow cytometry assays showed that fexofenadine and terfenadine did not affect the binding of TNF-α to TNFR1 in vitro and to the cell surface, although anti-TNF antibody completely blocked the binding of TNF to the cell surface (FIGS. 4E-G). These findings led us to propose that fexofenadine may have an additional unidentified target that mediates its anti-TNF activity through a previously unrecognized mechanism. To address this issue, we devoted significant efforts to isolate protein binding partners of fexofenadine. After failure with several approaches, including labelling and biochemical copurification, implementation of drug affinity responsive target stability (DARTS) assay proved successful. We first mixed cell lysate with fexofenadine or terfenadine for 1 hour and protease was added for 15 min. The digested proteins were separated by sodium dodecyl sulfate-polyacrylamide gel electrophoresis (SDS-PAGE), followed by Silver staining (FIG. 5A), and a band with the molecular weight of ˜80 kDa was found to be protected by fexofenadine and terfenadine. This band was excised from an accompanying Coomassie blue stained gel for protein identification by mass spectrometry (FIG. 5B), which led to the identification of PLA2G4A encoding cPLA2 (FIG. 5C) and IKBKB encoding IKK-β as potential candidates. Both cPLA2 and IKK-β have appropriate molecular weights and are known to be the critical mediators of inflammation. To determine whether both cPLA2 and IKK-β are the targets of fexofenadine and terfenadine, we performed Western blot of DARTS samples with which a series of protease to cell lysate ratios were implemented (FIG. 5D), and found that fexofenadine and terfenadine protected cPLA2, but not IKK-β, clearly indicating that cPLA2, but not IKK-β, is a novel target of fexofenadine and terfenadine.

In order to further confirm the associations of cPLA2 with fexofenadine, we employed the cellular thermal shift assay (CETSA), which allows for quantification of the change in thermal denaturation temperature of a target protein under different conditions, including those of varying temperature and concentrations of drug of interest. Both fexofenadine and terfenadine, particularly fexofenadine, prevent denaturation of cPLA2 and kept more cPLA2 in the soluble condition under several temperatures, strikingly obvious at 49° C., compared with DMSO (FIG. 5E, top). The melt curve showed a significant shift and obvious change of Tm in the presence of fexofenadine and terfenadine (Tm for control DMSO, terfenadine and fexofenadine are 46.09° C., 49.08° C. and 51.99° C., respectively) (FIG. 5E, bottom). Performance of CETSA at 49° C. with different dosages of drugs revealed that fexofenadine and terfenadine prevented cPLA2 denaturation in a dose-dependent manner, with the EC50 of 1.025 e-007 and 1.449 e-009, respectively (FIG. 5F).

To further characterize the interactions between fexofenadine and cPLA2, we performed both induced-fit docking (IFD) and molecular dynamics (MD) simulations. From IFD simulation, both fexofenadine and terfenadine core structures were predicted to stabilize at the mitogen-activated protein kinases (MAPK) phosphorylation site of cPLA2 at Ser-505 (FIG. 5G). Docked poses of fexofenadine in cPLA2 reveal that the fexofenadine-binding region was lined by Ser-505 and several residues close to Ser-505 (FIG. 5H). Fexofenadine was predicted to be involved in two hydrogen bonding interactions with Ser-505.

The predicted binding-region and hydrogen bonding interaction for terfenadine were nearly the same as fexofenadine (FIG. 5I). MD simulations, as a complement to IFD simulation, showed that fexofenadine core structure was majorly stabilized into the binding site predicted by IFD. The protein backbone root mean square deviation (RMSD) deviated up to about 4 A in the first 3 ns then remained relatively stable until the end of the simulation period, reflecting a relatively stable protein conformation. There was no significant turn-over in the fexofenadine binding pose as the fexofenadine RMSD deviated no more than 2 Å from the initiation of simulation (FIG. 14a). For the cPLA2-terfenadine binding complex, both the binding pocket of cPLA2 and the binding pose of terfenadine showed no significant steric changes with only slight fluctuations on RSMD values after 2 ns (FIG. 14b).

Fexofenadine inhibits TNF activity through binding to the catalytic domain 2 of cPLA2 and the inhibition of the phosphorylation of cPLA2 on Ser-505. cPLA2 contains several domains critical for its functions, including Ca2+ binding domain (C2D), catalytic domain 1 (CD1) and catalytic domain 2 (CD2) (FIG. 5J). We sought to identify the domain by which fexofenadine targets to cPLA2. For this purpose, we generated serial N-terminal and C-terminal deletion mutants and tested their interactions with fexofenadine by use of DARTS (FIG. 5J). Similar to the protective effect seen with intact cPLA2, fexofenadine retained protective effects for mutants with N-terminal C2D deletion (i.e., cPLA2 (126-750) and further deletion of CD1 (i.e., cPLA2(406-750)), indicating that the CD2 domain is the binding domain of fexofenadine. Indeed, fexofenadine did not show any protective effects on mutants lacking the CD2 domain (i.e., cPLA2 (1-479) and cPLA2 (1-144)). Collectively, these sets of assays identify the CD2 domain of cPLA2 as the binding domain of fexofenadine.

Interestingly, both IFD and MD simulations indicated that Ser-505, which is located in the CD2 domain of cPLA2, is the critical amino acid for the interactions between fexofenadine and cPLA2. We next determined whether the substitution of Ser-505 with Ala through the site-directed mutagenesis affected the binding of fexofenadine to cPLA2. DARTS assay clearly demonstrated that fexofenadine lost its protective effect on this point mutant of cPLA2 (FIG. 5K), further demonstrating that Ser-505 is the critical amino acid required for fexofenadine targeting to cPLA2.

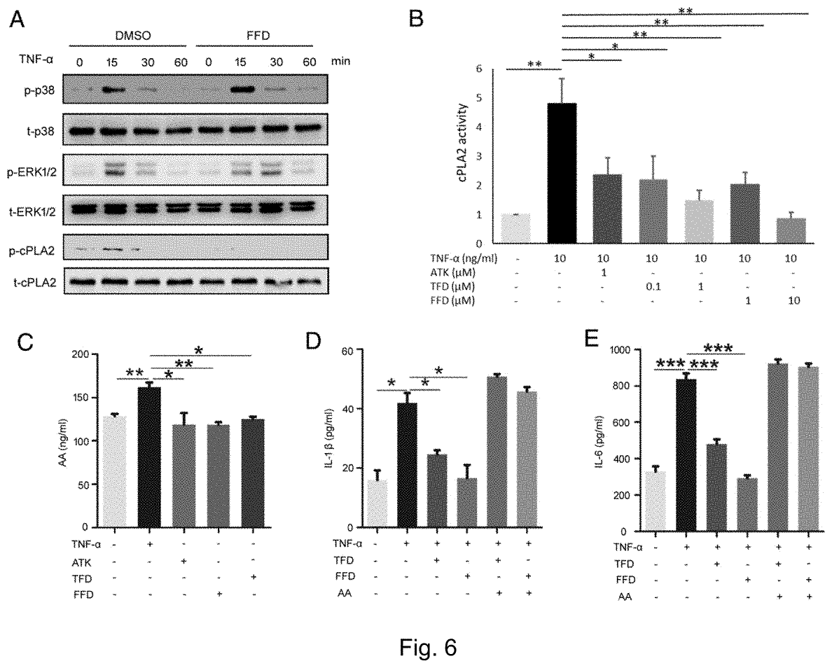

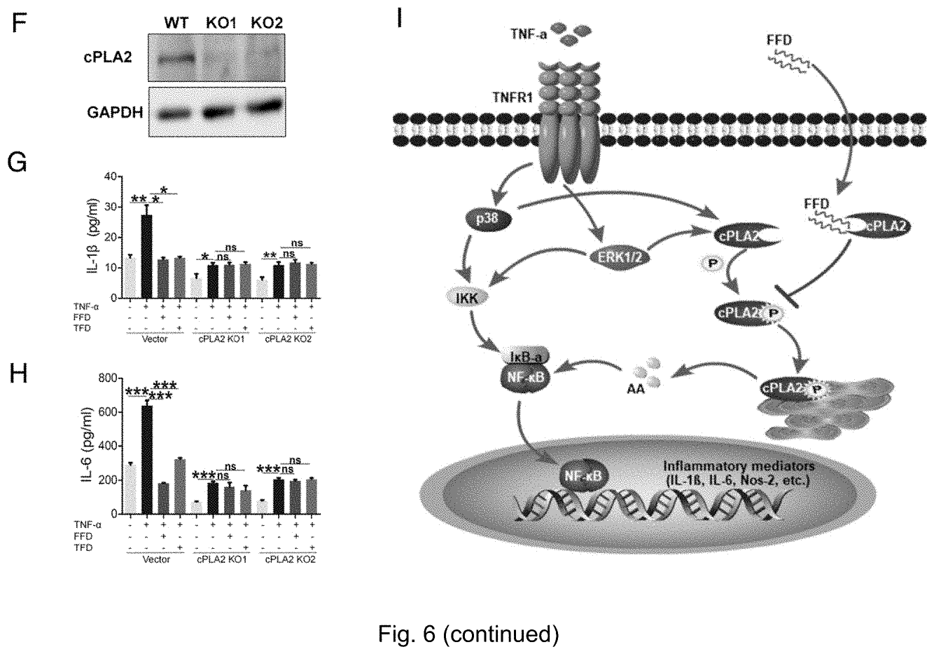

It is well established that the phosphorylation of cPLA2 on Ser-505 by upstream kinases p-p38 and p-REK1/2 is required for its enzymatic activity. Since Ser-505 is an essential amino acid of the binding site for fexofenadine targeting to cPLA2, we next examined whether fexofenadine affected TNF activated phosphorylation of cPLA2 on Ser-505 (FIG. 6A). As expected, TNF-α activated the phosphorylation of p38 and ERK1/2 as well as cPLA2 in BMDMs. Fexofenadine treatment did not affect the phosphorylation of p38 or ERK1/2, but abolished the phosphorylation of cPLA2 on Ser-505 (FIG. 6A). These results provide additional evidence that fexofenadine directly targets to cPLA2, without affecting its upstream mediators in the TNF-activated cPLA2 inflammatory pathway.

As mentioned earlier, the phosphorylation of cPLA2 on Ser-505 is required for its enzymatic activity; accordingly, we assessed whether Fexofenadine affected the activity of cPLA2. Similar to arachidonyl trifluoromethyl ketone 27 (ATK), a known cPLA2 inhibitor used here as a positive control, both fexofenadine and terfenadine completely abolished TNF-α induced cPLA2 activity and their inhibitions of cPLA2 activity are dosage dependent (FIG. 6B). In addition, ATK also inhibited TNF-α-induced cytokine release, but to a lesser degree, when compared with fexofenadine (FIG. 15a).

Inflammatory conditions, including elevated TNF-α, promote the cPLA2 translocation to intracellular phospholipid membrane. The major function of cPLA2 is to promote phospholipid hydro-lysis to produce AA, which, in turn, activates NF-κB-mediated inflammation. Accordingly, we examined whether fexofenadine and terfenadine inhibited TNF-α induced AA production, the data in FIG. 6C revealed that this was the case. Moreover, as shown in FIGS. 6D-E, supplementation of medium with AA eliminated the inhibitory influence of fexofenadine on TNF-α induced release of inflammatory cytokines IL-1β and IL6, suggesting that inhibition of AA production by fexofenadine is contributory to its anti-TNF-α activity. In addition, although fexofenadine inhibited stimulation of the TNF-α-activated NF-κB reporter gene, fexofenadine did not constrain AA activation of the NF-κB reporter gene (FIG. 15b), indicating that fexofenadine exerts it role upstream of AA production in NF-κB signaling. To further define the importance of cPLA2 in mediating fexofenadine's anti-TNF-α activity. We deleted PLA2G4A gene using the CRISPR-Cas9 technique (FIG. 6F). This technique produced near complete deletion of cPLA2 (FIG. 6F). Importantly, fexofenadine-mediated inhibition of TNF-α activity was entirely or almost entirely lost in cPLA2 knockout cells (FIGS. 6G-H). Further, fexofenadine lost its inhibition of TNF-α-activated NF-κB reporter gene in cPLA2 knockout cells. Re-establishing expression of cPLA2 by transfecting cPLA2 knockout cells with a cPLA2 expression plasmid reinstated fexofenadine's anti-TNF/NF-κB activity. However, transfection of cPLA2 knockout cells with an expression plasmid encoding a cPLA2 point mutant Ser-505-Ala (cPLA2 S505A), which inactivates cPLA2 enzymatic activity and fails to produce AA, could not rescue fexofenadine's anti-TNF/NF-κB activity (FIG. 15c). Taken together, these findings indicate the dependence of fexofenadine's anti-TNF/NF-κB activity on cPLA2 and cPLA2-mediated AA generation.

Discussion

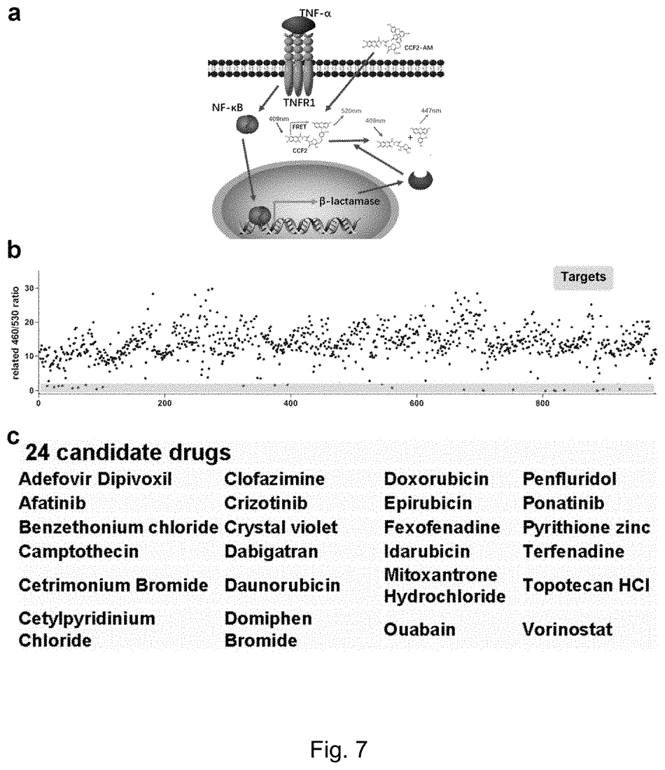

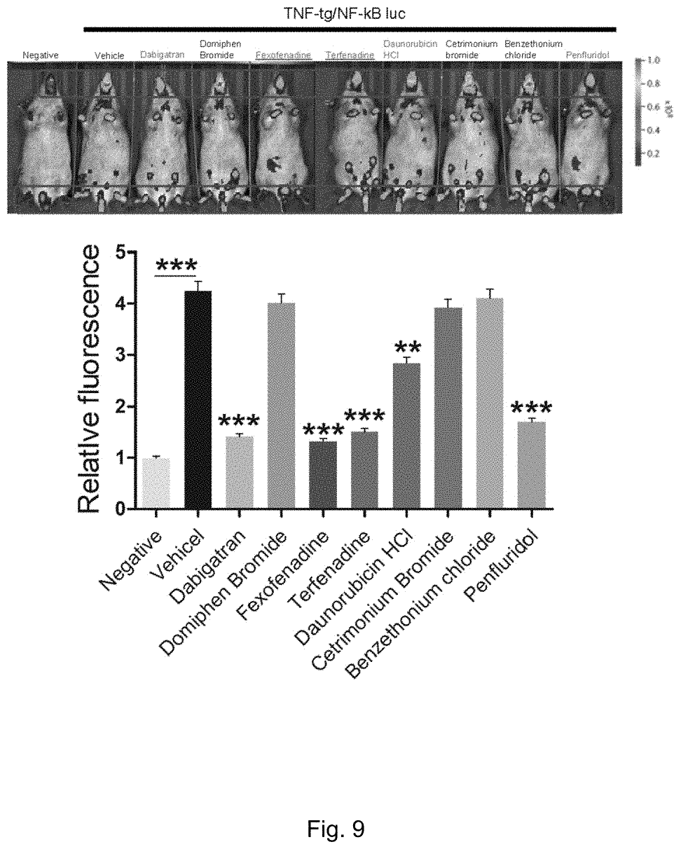

In this study, we performed three rounds of screening using an FDA-approved drug library, and isolated fexofenadine, a selective histamine receptor 1 antagonist, as a novel TNFI (FIGS. 7-10). Comprehensive evidences, including RNA-seq, transcription factor enrichment analysis, downstream cytokine expression and release, NF-κB nuclear translocation and activity, osteoclastogenesis as well as in vivo reporter and transgenic mice, validated fexofenadine's anti-TNF-α activities (FIG. 1, FIGS. S10-12). Subsequent in vivo animal models, including TNF-tg, and collagen-induced arthritis, demonstrated that fexofenadine is therapeutic against inflammatory arthritis to a degree better than, or at least as good as, the current small molecule drugs for treating rheumatoid arthritis (FIGS. 2 and 3). Intriguingly, fexofenadine exhibited better anti-TNF-α effects in the therapeutic treatment strategy than that in the preventive TNF-tg model (FIG. 2), suggesting that fexofenadine may exert a preferential effect during the progression of disease. Terfenadine also yielded beneficial effects in inhibiting TNF-α activity in vitro, but has adverse clinical effects. Fexofenadine, however, is not known to produce the significant health risks associated with terfenadine treatment and can therefore be used for treating chronic TNF-α-associated disease. Fexofenadine demonstrates features that compare favorably to established biologics such as etanercept (Enbrel) and adalimumab (Humira). For example, all currently marketed anti-TNF therapies bind to TNF-α and inhibit its binding to TNF receptors; in contrast to these upstream inhibitors, fexofenadine targets the downstream cPLA2 mediator of TNF-α signaling. In addition, it also targets H1R1. Due to this alternate mechanism of action, fexofenadine may be effective for the patients who fail to respond to current TNF-α blockers. As a well-tolerated and generically available oral OTC drug, fexofenadine's safety, convenience and cost-effectiveness it can be used for the clinical treatment of inflammatory autoimmune diseases, particularly rheumatoid arthritis.

Fexofenadine is known to be a highly selective antagonist to H1 receptor 1. Surprisingly, suppression of H1 receptor 1 does not affect fexofenadine-mediated anti-TNF-α activity. In addition, an additional seven known H1R1 inhibitors do not have anti-TNF activity (FIG. 4). Although fexofenadine potently inhibits TNF-α signaling in vitro and in vivo, it does not affect the binding of TNF-α to its receptors or cell surface, clearly different from clinically used TNFIs (FIG. 4). through combined use of drug affinity responsive target stability assay, proteomics, CETSA, IFD and MD, we identified cPLA2 as a previously unrecognized target of fexofenadine. Fexofenadine binds to catalytic domain 2 and inhibits the phosphorylation of cPLA2 on Ser-505. Further, deletion of cPLA2 abolished fexofenadine inhibition of TNF-induced AA production and downstream cytokine release (FIGS. 5 and 6). A proposed model for explaining the anti-TNF activity of fexofenadine through directly targeting the cPLA2 pathway is shown in FIG. 61. TNF-α binds to TNFR1 and activates p38 and ERK1/2, followed by the phosphorylation of cPLA2 on Ser-505. Phosphorylated cPLA2 then translocates from the cytosol to hydrolyze membrane phospholipids, leading to the production of AA. AA, in turn, actives NF-κB, leading to cytokine release and inflammation. Dissimilar to current TNF inhibitors that disturb TNF/TNFR interactions at the initiation of the signaling cascade, fexofenadine diffuses into the cells and directly binds to cPLA2 and inhibits its phosphorylation on Ser-505, followed by the inhibition of cPLA2/AA/NF-κB inflammatory pathway. It is also noted that fexofenadine inhibited the phosphorylation of NF-κB p65 upstream mediator IκB-α in vivo (data not shown), suggesting effects of fexofenadine on canonical TNF/IκB/NF-κB pathways, possibly through the cross-talk with the TNF/cPLA2/NF-κB pathway.

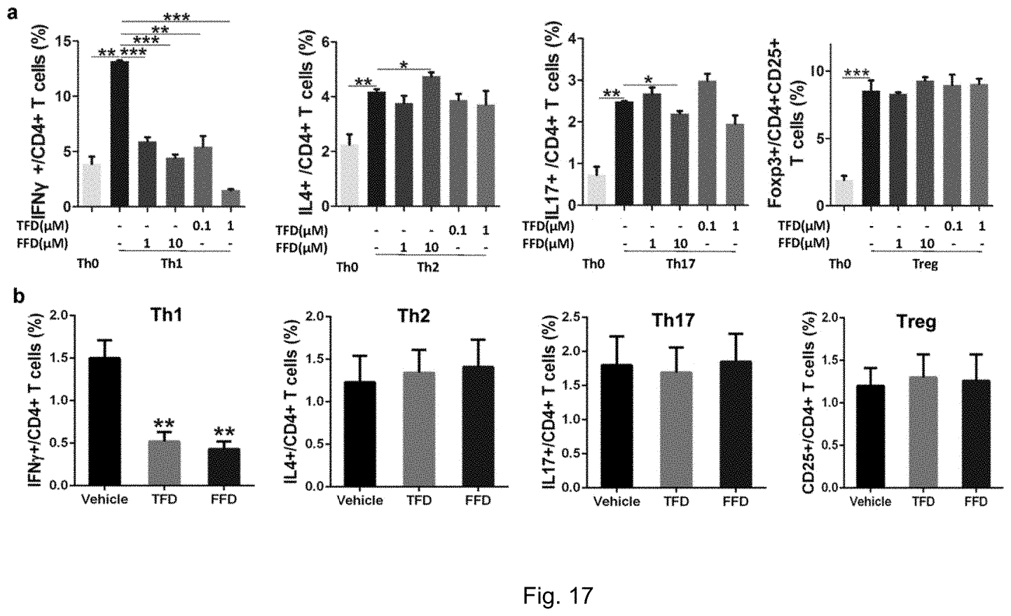

We found that both fexofenadine and terfenadine significantly inhibited inflammatory M1 macrophages, while markedly increased anti-inflammatory M2 macrophages (FIG. 16). Furthermore, fexofenadine and terfenadine significantly suppressed the differentiation of IFNγ-positive Thl subpopulation in vitro and in vivo, whereas negligible effects were observed with regard to the differentiation of Th2, Th17 and regulatory T cells (FIG. 17). We demonstrate that fexofenadine is therapeutic against inflammatory arthritis spontaneously developed in TNF-tg mice (FIG. 2) and fexofenadine-mediated anti-TNF-α activity depends on cPLA2 (FIGS. 6F-H), but not histamine H1 receptor (FIG. 4); however, its anti-histaminic action may also contribute to its therapeutic effects in inflammatory arthritis.

These results demonstrate that Similar to TNF-α, cPLA2 is also known to play an important role in regulating autoimmune diseases. This study identifies fexofenadine as an inhibitor of TNF-α signaling. This study also identifies cPLA2 as a new target of fexofenadine. Our data also identifies fexofenadine as a novel antagonist of cPLA2, suggesting that fexofenadine can also be used for treating various cPLA2-associated diseases, including autoimmune diseases.

Materials and Methods

In vitro and in vivo screen of FDA-approved drug library. NF-κB-bla THP-1 cell line in which NF-κB beta-lactamase reporter gene was stably integrated, RAW 264.7 macrophages in which NF-κB luciferase reporter gene was transiently transfected, and TNF-tg:NF-κB-Luc double mutant reporter mice were employed to screen the drug library.

In vitro and in vivo assays for examining the blockade of TNF actions by fexofenadine. RNA-seq, transcription factor enrichment analysis, downstream cytokine expression and release, NF-κB translocation and activity, osteoclastogenesis with BMDMs and RAW264.7 macro-phages, as well as in vivo reporter mice.

In vivo assays for defining the anti-inflammatory activity of fexofenadine using various animal models. Administration of fexofenadine, terfenadine or clinically used positive controls into TNF-Tg mice and CIA of DBA/1 mice.

Identification and characterization of the binding of fexofenadine to cPLA2. Drug affinity responsive target stability assay, proteomics, CETSA, information field dynamics and MD; solid-phase binding and flow cytometry was used to examine the effects of fexofenadine on TNF/TNFR interactions.

Assays for examining fexofenadine inhibition of cPLA2 as well as the dependence on cPLA2 of fexofenadine's anti-TNF activity. Phosphorylation of p38, Erk1/2 and cPLA2 by TNF-α, activation of cPLA2 activity and AA production by TNF-α, and their inhibition by fexofenadine, dependence of fexofenadine-mediated inhibition of TNF-α on the presence and activity of cPLA2 and AA production were determined.

Screening FDA approved drugs in vitro. NF-κB-bla THP-1 cell line (K1662, Invitrogen) was cultured in 96-well plates and FDA approved drugs (10 μM, L1300, Selleckchem) were added and cultured overnight. Next day, cells were incubated with TNF-α (10 ng/ml, PHC3015, Invitrogen) for 6 hours and LiveBLAzer™ FRET-B/G Loading Kit with CCF2-AM (K1095, Invitrogen) was used to detect the β-lactamase activity via SpectraMax i3x system (5025027A Molecular Devices) with excitation wavelength at 409 nm and emission wavelengths at 520 nm or 470 nm. This process was performed in triplicate. Subsequently, the drugs from first screening that could inhibit TNF-α induced NF-κB activity underwent further screening by NF-κB luciferase assay. After transfection with NF-κB luciferase reporter gene and Renilla plasmids for 8 hours, cells were treated with drugs (10 μM) overnight, followed by TNF-α (10 ng/ml) stimulation for 6 hours, then bioluminescence was measured by Dual-Luciferase® Reporter Assay kit (E1910, Promega). The data were analyzed by Graphpad Prism software (GraphPad Software, San Diego, Calif.).

Screening and confirming the drugs' anti-TNF activity in vivo. TNF-tg mice and NF-κB luc mice were purchased from Jackson Laboratory and housed in Skirball Animal Facility of New York University Langone Medical Center. All animal experiments have been approved by Institutional Animal Care and Use Committee (IACUC) of New York University School of Medicine. We generated the TNF-tg/NF-κB-Luc mice by mating TNF-tg mice with NF-κB-Luc mice. After genotypes were confirmed by genotyping and bioluminescence via IVIS, the TNF-tg/NF-κB-Luc mice were orally treated daily with one of the 8 drugs selected in screen two for 7 days. The luciferase activity was detected by IVIS 15 minutes after the injection of D-Luciferin (LUCK-1G, Gold biotechnology).

Primary bone marrow derived macrophages (BMDMs) extraction. BMDMs were isolated from C57BL/6 and TNF-tg mice. After mice were sacrificed by cervical dislocation, the tibia and femur were isolated and both ends of the bones were cut to open the medullary cavity. Bone marrow cells were collected by centrifuge at 13000 g for 90 seconds and seeded in 6-well plates. Prior to their use in experiments, M-CSF (10 ng/ml, 576406, Biolegend) was added for 6 days to the BMDM culture medium.

qRT-PCR. Total RNA was extracted by RNeasy plus mini kit (74106, QIAGEN) and cDNA was synthesized using SuperScript® Reverse Transcriptase (M314C, promega Corporation). SYBR® Green PCR Master Mix (4309155, Applied Biosystems) was used to perform quantitative real-time PCR (qRT-PCR) and the reaction was performed on a StepOnePlus™ real-time PCR Systems (Applied Biosystems). The mRNA expression level of target genes was calculated by ΔΔCT and fold changes of mRNA levels were normalized to GAPDH.

ELISA. Cytokine levels of IL-1β and IL-6 in cell culture supernatants or sera from mouse models were detected by sandwich ELISA according to product specifications in commercial ELISA kits (IL-1β 88-7013, Invitrogen; IL-6: 88706476, Invitrogen). Cell cultural supernatants were collected following treatment with drugs for 48 hours. Sera were separated from whole blood by centrifuge of freshly collected blood at 3000 rpm for 10 minutes.

Western blot. After protein samples were prepared, samples were separated by SDS-PAGE and transferred to a nitrocellulose (NC) membrane (162-0115, BIO-RAD) using a wet transfer system. Membrane was blocked in 5% (w/v) non-fat milk in TBST for half an hour at room temperature, followed by incubation with appropriate primary antibody overnight at 4° C. After washing, appropriate secondary antibody was added for 1 hour at room temperature. The bands on the membrane were developed by chemiluminescent (ECL) substrate and visualized by GelDoc system.

RNAseq and Transcription factors enrichment analysis. BMDMs were incubated with Fexofenadine (10 μM, 53208, Selleckchem) or Terfenadine (1 μM, T9652, Sigma) with or without TNF-α (long/mL) for 24 hours. Total RNA was extracted by RNeasy Mini Kit (74106, Qiagen), and gene expression profiling analyzed by RNA-seq was performed by the NYU Genome Technology Center for RNA sequencing (Illumina HiSeq4000 Sequencing, HiSeq 4000 Single Read 50 Cycle Lane). In addition, TNF-α induced genes that were suppressed by FFD/TFD were used for transcription factor enrichment analysis with TFactSⁱ.

Osteoclastogenesis. BMDMs were obtained as described above and the resultant preosteoclasts were cultured in medium supplemented with 10 ng/ml TNF-α and 100 ng/ml RANKL in the absence or presence of Fexofenadine or Terfenadine for 4 days. The cells were fixed with formalin and stained for tartrate-resistant acid phosphatase (TRAP) with a TRAP solution containing 100 mM sodium acetate buffer (pH 5.0), 50 mM sodium tartrate, 0.1 mg/ml sodium naphtol AS-MX phosphate, 0.6 mg/ml Fast Violet LB, and 0.1% Triton X-100. TRAP-positive cells appeared dark red and TRAP-positive multinucleated cells containing more than three nuclei (TRAP-MNCs) were visualized using light microscopy.

Nuclear translocation and DNA binding activity of NF-κB. BMDMs were treated with or without Fexofenadine (10 μM) or Terfenadine (1 μM) overnight followed by 6 hour incubation with TNF-α (long/mL). Immunofluorescence was performed to test the location of p65 (4764s, Cell Signaling Technology). The cytoplasmic and nuclear protein fractions were extracted with a hypotonic solution and hypertonic solution. p65 (4764, Cell Signaling Technology), Lamin B (sc-374015, Santa Cruz Biotechnology) and GAPDH (2118, Cell Signaling) were detected in cellular fractions by Western blot. The total proteins were extracted for analysis of p65 DNA binding activity by TransAM® NFκB p65 ELISA kit (40096, Active motif).

hTNF-tg Mouse Model

The human TNFα gene was microinjected into C57BL/6 background mice to generate the human TNF transgenic (TNF-tg) mouse, which highly express TNFα and spontaneously develops arthritisZ. For prevention assessments, oral treatments of Fexofenadine (10 mg/kg), Terfenadine (50 mg/kg), and MTX (2 mg/kg, serving as a positive control) were started at 8-weeks of age and continued for a total of 13 weeks. Treatments were stopped at the 17-weeks-time point and resumed at the 19-weeks-time point to observe the response of inflammatory arthritis progression to Fexofenadine or Terfenadine. For treatment assessment, oral delivery of Fexofenadine (0.4, 2, 10 mg/kg), Terfenadine (2, 10, 50 mg/kg), and MTX (2 mg/kg) was initiated when the average swelling score reached approximately 8 points and treatment continued for a total of 8 weeks. Six mice in each group. Swelling scores were assessed weekly in accordance with the scoring system offered by the company. The swelling scores were taken as the sum of the scores from digits, paws, wrists and ankles with the highest possible score for each mouse at 24. The score system is: 20 digits (0=normal, 0.2=one or more swollen joints), 4 paws (0=normal, 1=noticeable swollen, 2=severe swollen), 2 wrists (0=normal, 1=noticeable swollen, 2=severe swollen), and 2 ankles (0=normal, 2=noticeable swollen, 4=severe swollen). At the end of treatment and observation, mice were sacrificed for collection of sera, spleens and ankles.

Collagen induced arthritis model. Eight-week old male DBA/1J mice were purchased from Jackson Lab. Emulsion of complete Freund's adjuvant (7001, Chondrex) and chicken type II collagen (20012, Chondrex) was intradermally injected at the site 1.5-2 cm distance from the tail base. For prevention assessment, oral treatments of Fexofenadine (10 mg/kg), Terfenadine (50 mg/kg), and MTX (2 mg/kg, serving as a positive control) were started at the 18th day after immunization and continued for a total of 48 days. For treatment assessment, treatments of Fexofenadine (0.4, 2, 10 mg/kg), Terfenadine (2, 10, 50 mg/kg), MTX (2 mg/kg) were orally delivered beginning when the average clinic score reached approximately 5 points; treatment continued daily for a total of 24 days. Eight mice in each group. Clinic score was recorded every other day based on the following score system³: 0=normal, 1=mild swelling involving ankle, wrist, or one digit, 2=mild swelling involving entire paw or more than two digits, 3=moderate swelling from the ankle/wrist to entire foot/paw and all digits, 4=severe swelling at the whole ankle/wrist, foot/paw and digits or ankylosing deformity. After the mice were sacrificed, sera and joints were collected for further detection.

Histology and analysis. Tissues were fixed in 4% formaldehyde, decalcified in 10% EDTA and embedded in paraffin. Serial 5 μm sections were cut and stained with hematoxylin and eosin (H&E). Images were obtained by digital microscope (Axio Scope A.1, Carl Zeiss, LLC) and scored by two independent observers. The ankles were scored on inflammation and pannus formation and articular cartilage damage. Inflammation was scored as follows: 0, Normal, 1, local inflammatory infiltration and 2, marked infiltration with lymphoid aggregates and edema. Pannus formation and articular cartilage damage were scored as follows: 0, Normal, 1, synovial proliferation adjacent to cartilage but no articular cartilage damage and 2, synovial proliferation and articular cartilage damage.

Micro-CT. Ankle tissues were fixed by 4% formaldehyde and stored in 70% ethanol. After fixation, tissues were scanned, at a resolution of 10.5 μm, by Scanco vivaCT40 cone-beam scanner (SCANCO Medical, Switzerland) with 55 kVp source and 145 μAmp current. The scanned images from each group were evaluated at the same thresholds to allow 3-dimensional structural reconstruction of each sample.

TRAP staining. For paraffin-embedded slides, the sections were firstly deparaffined in a xylene and ethanol gradient, and then stained with TRAP staining solution mix for 60 minutes at 37° C. and counterstained with methyl green for 5 minutes, followed by dehydrating through graded ethanol and xylene. For live cultured cells, cells were firstly fixed with 10% formaldehyde for 10 minutes at room temperature prior to staining. The osteoclasts were revealed as multinucleated cells stained red violet against the green background. Images were taken by light microscope (Axio Scope A.1, Carl Zeiss, LLC).

Safranin O staining. After the paraffin-embedded knee and ankle sections were deparaffined in a xylene and ethanol gradient, sections were stained with 2% hematoxylin (23412, MilliporeSigma) for 5 min, 1.0% Safranin O (S8884, Sigma-Aldrich) for 60 min, and counterstained with 0.02% Fast Green (F7258, Sigma-Aldrich) for 1 minute. Stained slides were dehydrated, cover slipped and photographed using a light microscope (Axio Scope A.1, Carl Zeiss, LLC).

DARTS assay. Drug affinity responsive target stability (DARTS) assay was performed based on previously reported methods⁴. In brief, cell lysate was extracted by M-PER™ Mammalian Protein Extraction Reagent (78501, Thermo Fisher), briefly centrifuged, and mixed with drugs or DMSO for 1 hour on a rotator. Pronase (P5147, Sigma-Aldrich) was added in the mixture for 15 minutes at room temperature. Digestion was stopped by 10 minute incubation with protease inhibitor cocktail on ice. Samples were boiled in SDS loading buffer for SDS-PAGE and Western blot.

CETSA assay. RAW264.7 cells were treated with FFD (10 μM)/TFD (1 μM) for 1 hour in a 37° C. incubator with 5% CO₂. Cells were harvested and divided equally into different tubes, after which the cells were heated to different temperatures for 3 minutes. After lysis via 3 repeated freeze-thaw cycles, samples underwent centrifugation at 20000 g and supernatants were collected for analysis. For the isothermal dose response, a series of ten-fold change concentrations (ranged from 0.001 μM to 100 μM) of FFD/TFD were used to treat cells and no variation in temperature was introduced. The remaining steps are the same as previously reported⁵.

Site-directed mutagenesis. PLA2G4A cDNA from Genscript was used as a template and Ser-505 was mutated to Ala using Q5® Site-Directed Mutagenesis Kit in accordance with the manufacturer's instructions (E0554, New England Biolabs). To specific, taking PLA2G4A cDNA clone from Genscript as a template, the base of T was mutated to G in the amino acid Ser-505 (TCT) which changed to Ala505 (GCT).

Construction of plasmids. The construction of cPLA2 mutant plasmids was based on serial C-terminal and N-terminal deletion of the amino acid sequences associated with functional structure. Each plasmid carried a flag tag. The constructions were: full length cPLA2 (1-750), cPLA2 (126-750), cPLA2 (406-750), cPLA2 (1-479), and cPLA2 (1-144).

cPLA2 activity assay. RAW264.7 cells were transfected with an expression plasmid encoding cPLA2. 24 hours later, the transfected cells were treated with TNF-α (10 ng/ml) and ATK (1 μM), or TFD (0.1 μM or 1 μM), or FFD (1 μM or 10 μM) overnight. Then the cells were collected and lysed. Cell lysate was used for measurement of cPLA2 activity using a commercial ELISA kit (765021, Cayman Chemical). Briefly, protein concentration of the cell lysate was measured by bicinchoninic acid assay and equal amounts of protein from each lysate sample were incubated with cPLA2 substrate (Arachidonoyl Thio-PC) at room temperature. One hour later, DTNB/EGTA was added to stop the enzyme catalysis, and then the absorbance was read at 414 nm using a plate reader (SpectraMax i3x, 5025027A, Molecular Devices).

Knock down of H1R1 by siRNA. BMDMs were transfected with siH1R1, or scrambled control siRNA (scRNAi) using lipofectamine 2000 (11668-019, Invitrogen) according to manufacturer's specifications. After 24 hours, supernatants and lysate were collected to detect cytokine secretion and protein expression, respectively.

Knock out of cPAL2 by CRISPR-Cas9. Knockout cells were generated in accordance with a previously published protocol. gRNA was inserted into the lentiCRISPR v2 vector. Packaging the inserted vector into lentivirus was completed by co-transfecting the lentiCRISPR v2, VSVG and ΔR6.7 in 293T cells. After packaging, we transfected the virus into RAW264.7 cells over 8 hours. Puromycin (2 μg/ml) was used to select the stably transfected RAW264.7 cells

Arachidonic acid detection. BMDMs were treated with TNF-α (10 ng/ml) with or without Fexofenadine/Terfenadine/ATK, after 48 hours the culture medium was collected and analyzed using an arachidonic acid ELISA kit (NBP2-66372, Novus Biologicals). The result was detected at 450nm by automatic plate reader (SpectraMax i3x system, 5025027A, Molecular Devices). ATK was implemented as a positive control.

Solid phase binding. To test whether Fexofenadine/Terfenadine affect the binding between TNF-α and TNFR1, solid phase binding assay was performed according to a previously described method⁷. Briefly, a 96-well ELISA plate was coated with 100 μL of 0.5 ng/μL TNFR1 at 4° C. overnight. The plate was washed with PBST 5 times, and 300 μL blocking buffer was added to each well followed by incubation at room temperature for 1 hour. After discarding the blocking buffer, 50 μL buffer containing drugs at concentrations ranging from 0.1 nM to 10⁵ nM were added to the plate for 1 hour at room temperature. Three wells incubated with bovine serum albumin (BSA) served as negative controls and three wells containing TNF-α were set as positive controls. Without washing, 50 μL buffer containing 10 ng biotin-labeled TNF-α was added to each well and incubated at room temperature for 2 hours. The plate was washed and 100 μL buffer containing streptavidin-HPR was added and incubated at room temperature for 30 minutes. After a final wash, 100 μL TMB buffer was added to each well and the reaction was stopped when the positive control group turned blue. The plate was read by automatic plate reader at 450 nm.

Flow cytometry. To test whether Fexofenadine/Terfenadine affected the binding between TNF-α and TNFRs expressed on cell surface, flow cytometry was performed. RAW 264.7 cells were seeded in a 12-well plate. After adding DMSO (control group), Fexofenadine (10 μM) or Terfenadine (1 μM) overnight, cells were stained according to manual specifications (NFTAO, R&D Systems). Then the samples were analyzed at NYU core facilities using a FACS Calibur cell analyzer with CellQuest software.

Spleen CD4⁺T cell differentiation. Inducing spleen naive CD4⁺T cell differentiation into T cell subsets was performed. Briefly, after preparing the splenic naïve CD4⁺T cells from wild-type 8-12 week old C57BL/6 mice, the following cytokines were added to induce naive CD4⁺T cell differentiation into T cell subsets: IL-2 (20 ng/mL), IL-12 (15 ng/mL), and anti-IL4 (5 μg/mL) for Thl; IL-2 (20 ng/mL), IL-4 (10 ng/mL), and anti-IFNγ (5 μg/mL) for Th2; IL-6 (20 ng/mL), TGFβ (3 ng/mL), anti-IFNγ (5 μg/mL), and anti-IL4 (5 μg/mL) for Th17; IL-2 (20 ng/mL), TGFβ (15 ng/mL), anti-IFNγ (5 μg/mL), and anti-IL4 (5 μg/mL) for Treg. Fexofenadine (1 or 10 μM) or Terfenadine (0.1 or 1 μM) were added and cells were cultured for 4 days without changing the medium.

Before collecting cells for fluorescence staining, 1 μL Golgi stop from Fixation/Permeabilization Solution Kit with BD GolgiPlug™ (555028,BD Biosciences) was added to each well and incubated in the plate for 4 hours. Cells were stained according to the kit specifications. For each T cell subtype, the combination of fluorescence dyes was as follows: FITC-CD4 (M1004502, Sungene) and Percp-cy5.5-IFNγ (45-7311-82, eBioscience) for Th1; FITC-CD4 and PE-IL4 (554389, BD pharmingen) for Th2; FITC-CD4 and PE-IL-17A (12-7177-81, eBioscience) for Th17; FITC-CD4, PE-CD25 (102008, Biolegend) and Alex-Fluo647-FoxP3 (51-5773-80, eBioscience) for Treg. Then the samples were analyzed at NYU core facilities using a FACS Calibur cell analyzer with CellQuest software.

Macrophage polarization. To test the influence of Fexofenadine/Terfenadine on macrophage polarization, subtype marker expressions were tested by qRT-PCR. Nos2 and IL-6 were used to indicate M1 macrophage while Argl and Mgll were used to indicate M2 macrophage. qRT-PCR primer sequences of the markers were: Argl 5′-3′: F-TGC CAA AGA CAT CGT GTA CAT TG (SEQ ID NO:1) and R-CTT CCC AGC AGG TAG CTG AAG (SEQ ID NO:2). Mgl1 5′-3′: F-CAG ATC CGT ATC TGT CTG GAT C (SEQ ID NO:3) and R-AGG TGG GTC CAA GAG AGG ATG (SEQ ID NO:4). Nos2 5′-3′: F-TGT TAG AGA CAC TTC TGA GGC TC (SEQ ID NO:5) and R-ACT TTG GAT GGA TTT GAC TTT GAA G (SEQ ID NO:6). IL-6 5′-3′: F-TTC CAT CCA GTT GCC TTC TTG (SEQ ID NO:7) and R: AGG TCT GTT GGG AGT GGT ATC (SEQ ID NO:8). INFγ (25 ng/mL) and LPS (250 ng/mL) were added to BMDMs to induce M1 polarization while IL-4(20 ng/mL) was used to induce M2 polarization, with or without adding FFD (1 or 10 μM) or TFD (0.1 or 1μM), and cells were cultured for 24 hours. mRNA were extracted by RNA Mini kit (74106, QIAGEN).

Assays to examine the in vivo effects of Fexofenadine on T cell subsets and macrophage differentiation. Frozen splenic tissue specimens from TNFα Tg mice treated with or without TFD or FFD were stained and analyzed dual immunofluorescence. All sections were permeabilized with 0.1% NP40 in PBS, and then blocked in 5% donkey serum for 30 min at RT, followed by incubated with anti-IFNg (Th1), anti-IL-4 (Th2), anti-IL17 (Th17) and anti-foxp3 (Treg) diluted in 5% donkey serum at 4° C. overnight. On the second day, after washed 3 times in PBS, the sections were incubated with Cy2-conjugated donkey anti-rat IgG for 30 min at RT. After wash, the sections were furthered stained with FITC conjugated anti-CD4 for I hour at RT. To determine the macrophage polarization, sections were double stained with anti-CD68 and iNOS (M1) or CD206 (M2). DAPI were used to stain the nuclei. The fluorescence intensity of cells was quantified by Image J software.

Protein Modeling and Preparation. The crystal structure of human cPLA2 (PDB ID: 1CJY, 2.5 Å) is available, however, there are several missing regions (residues 1 to 12, 406 to 414, 433 to 459, and 499 to 538) in this crystallized protein that were not resolved in the X-ray diffraction¹⁰. The missing structural regions were modeled into chain A of 1CJY using Modeller v9.20 (Andrej Sali Lab, UCSF, CA, USA, 2018). The generated homology model of 1CJY was further equilibrated and refined using 50 ns of all atom MD simulations by Desmond v5.6. The refined protein structure was then prepared using Protein Preparation Wizard implemented in Maestro v11.1 (Schrodinger, Inc., NY, USA, 2017) for docking simulation.

Ligand Docking and Molecular Dynamics (MD) Simulation. The 3D structures of Fexofenadine and its parent drug Terfenadine were built using Maestro v11.1 and energy minimized using the Macromodel v11.5 module. The ligands were then prepared with Ligprep v4.1 module to generate low-energy 3D structures. Since the cell-based study implicated that the impeded phosphorylation on residue Ser-505 may be a key mechanism involved in the inhibitory effect of Fexofenadine on cPLA2, a docking grid (25 Å) was generated by selecting Ser-505 as centroid. Flexible docking of Fexofenadine and Terfenadine into cPLA2 was carried out using the XP (extra precision) mode by Glide v7.4 (Schrodinger, Inc., NY, USA, 2017). Taking the flexibility of receptor and ligand into consideration to get optimal binding simulation between ligand and receptor, induced-fit docking (IFD) by Glide v7.4 was performed. The ligand binding pose with the lowest predicted ligand binding free energy obtained from the Glide XP docking processing was subjected to IFD simulation following the default IFD protocol. The Glide Emodel value was ranked to identify the best docked pose among multiple conformations¹¹. To validate the prediction of the binding, the docked cPLA2-Fexofenadine complex and cPLA2-Terfenadine complex with the best Glide Emodel values were subsequently subjected to short molecular dynamics simulation with Desmond MD system v5.6 (D. E. Shaw Research, NY, USA, 2018; Schrödinger, Inc., NY, USA, 2018). Predefined TIP3P water model was used and counter Na+/Cl− ions were added to balance the system charge. Periodic boundary conditions were set up and the buffer distance between box wall and the protein-ligand complex was set to be greater than 15 Å to avoid direct interaction of the complex with its own periodic image. After establishing the solvated system, MD simulation was carried out in the NPT ensemble using OPLS 2005 force field. The temperature and pressure were retained at 300 K and 1 atmospheric pressure. A short equilibration phase simulation was involved using default Desmond protocol, followed by running the 10,000 ps (10 ns) MD simulation for the equilibrated complex system. Schrödinger simulation interactions diagram (SID) was used to evaluate the interaction between ligand and protein, and the root mean square deviation (RMSD) of the ligand-receptor complex was calculated to evaluate if there are conformational changes of the protein or internal fluctuations of the ligand. The RMSD was calculated for all frames in the simulation trajectory, with respect to the first frame as the reference frame. All calculations mentioned above were performed on a 6-core Xeon processor except MD jobs which were performed on a Nvidia GPU.

Statistical Analysis

Data were reported as the means with standard errors. Comparisons among the treatment groups were performed by repeated measures in SPSS software (IBM, Armonk, N.Y., USA) or unpaired t-tests/one-way ANOVA in Graphpad software (GraphPad Software, San Diego, Calif.). P value <0.05 was statistically significant at two-sides.

### EXAMPLE 2

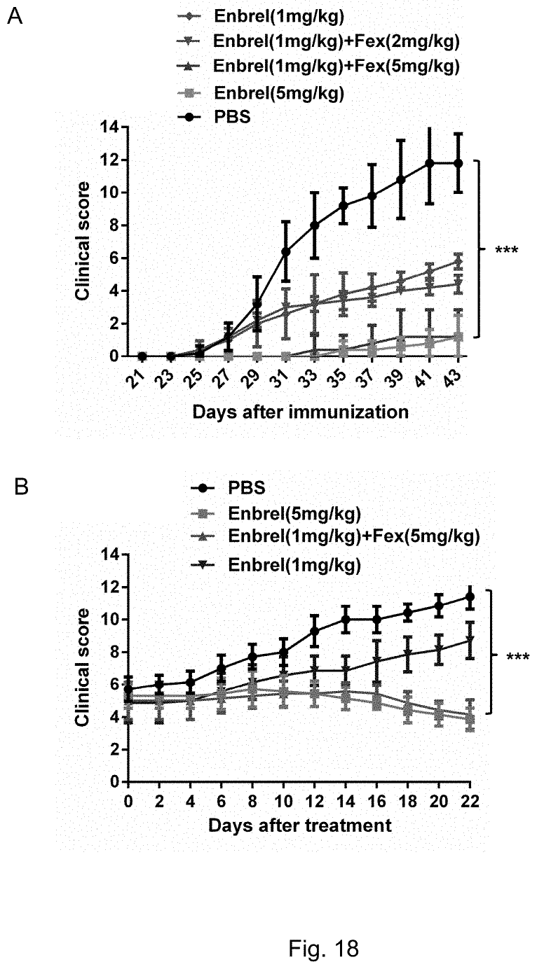

This example demonstrates that Fex can help to reduce the dosage of other TNF-α inhibitors in the treatment of inflammatory autoimmune diseases. In this example, the effects of etanercept/fexofenadine combination in collagen-induced arthritis (CIA) mice were studied. Mice were immunized with CII in CFA and boosted 21 days later to induce CIA, and the prevention started at 0 day after the second immunization. CIA mice with arthritis severity score above 5 after the second immunization were selected and treated (i.p. injection) with PBS, etanercept (5 mg/kg), etanercept (1 mg/kg) or fexofenadine(5 mg/kg)+etanercept (1 mg/kg) once a week for 4 weeks. Arthritic symptoms and histological stainings were evaluated. As shown in FIG. 18, combination therapy significantly decreased arthritis score in the prevention model (A) and the therapeutic model (B) when Fex was used with etanercept. N=7 per group. *P<0.05 versus vehicle, **P<0.01 versus vehicle, ***P<0.001 versus vehicle.

While the present invention has been described through illustrative embodiments, routine modification will be apparent to those skilled in the art and such modifications are intended to be within the scope of this disclosure.

What is claimed is:

1. A method of treating an inflammatory autoimmune disease comprising administering to a subject in need of treatment, a composition comprising a therapeutically effective amount of Fexofenadine and/or Terfenadine.

2. The method of claim 1, wherein the inflammatory autoimmune disease is inflammatory bowel disease or rheumatoid arthritis.

3. The method of claim 1, wherein the subject is not suffering from allergies.

4. The method of claim 1, wherein the subject has previously not responded to Etanercept, Infliximab, Adalimumab, Certolizumab, and Golimumab.

5. The method of claim 1, further comprising administering to the individual Etanercept, Infliximab, Adalimumab, Certolizumab, or Golimumab.

6. The method of claim 5, wherein either fexofenadine or terfenadine, or one of Etanercept, Infliximab, Adalimumab, Certolizumab, and Golimumab, is at a sub-therapeutic dose.

7. The method of claim 6, wherein the dose of fexofenadine or terfenadine is less than half of an effective therapeutic dose.

8. The method of claim 6, wherein the dose of one of Etanercept, Infliximab, Adalimumab, Certolizumab, and Golimumab is less than half of an effective therapeutic dose.

9. The method of claim 6, wherein both fexofenadine or terfenadine, and one of Etanercept, Infliximab, Adalimumab, Certolizumab, and Golimumab, are at a sub-therapeutic dose.

10. The method of claim 4, wherein the fexofenadine is administered to the subject while continuing treatment with Etanercept, Infliximab, Adalimumab, Certolizumab, and Golimumab.

11. A composition comprising a synergistic combination of i) fexofenadine or terfenadine, and ii) one of Etanercept, Infliximab, Adalimumab, Certolizumab, and Golimumab, wherein i) and/or ii) are present at a sub-therapeutic dose.

12. A kit for treatment of inflammatory autoimmune disease comprising in the same or different packaging, i) fexofenadine and ii) one of Etanercept, Infliximab, Adalimumab, Certolizumab, and Golimumab, wherein i) and/or ii) is/are present in a subtherapeutic dose, and optionally instructions for use of i) and/or ii).

13. The kit of claim 12, wherein the inflammatory autoimmune disease is inflammatory bowel disease or rheumatoid arthritis.


SEQUENCE LISTING (11 sequences)

SEQ ID NO: 1 - DNA, 23 bp, Artificial Sequence
  primer
tgccaaagacatcgtgtacattg

SEQ ID NO: 2 - DNA, 21 bp, Artificial Sequence
  primer
cttcccagcaggtagctgaag

SEQ ID NO: 3 - DNA, 22 bp, Artificial Sequence
  primer
cagatccgtatctgtctggatc

SEQ ID NO: 4 - DNA, 21 bp, Artificial Sequence
  primer
aggtgggtccaagagaggatg

SEQ ID NO: 5 - DNA, 23 bp, Artificial Sequence
  primer
tgttagagacacttctgaggctc

SEQ ID NO: 6 - DNA, 25 bp, Artificial Sequence
  primer
actttggatggatttgactttgaag

SEQ ID NO: 7 - DNA, 21 bp, Artificial Sequence
  primer
ttccatccagttgccttcttg

SEQ ID NO: 8 - DNA, 21 bp, Artificial Sequence
  primer
aggtctgttgggagtggtatc

SEQ ID NO: 9 - DNA, 30 bp, Homo sapiens
tcttatccactgtctcctttgagtgacttt

SEQ ID NO: 10 - DNA, 30 bp, Homo sapiens
tcttatccactggctcctttgagagacttt

SEQ ID NO: 11 - protein, 10 aa, Homo sapiens
VLGVSGSQSR